(19) United States
(12) Patent Application Publication
(10) Pub. No.: US 2023/0371872 A1
(43) Pub. Date: Nov. 23, 2023

(54) METHOD AND SYSTEM FOR QUANTIFYING ATTENTION

(71) Applicant: InnerEye Ltd., Herzeliya (IL)

(72) Inventors: Yuval HARPAZ, Rehovot (IL); Amir B. GEVA, Herzliya (IL); Leon Y. DEOUELL, Tel-Aviv (IL); Sergey VAISMAN, Ramat-Gan (IL); Yaar SHALOM, Tel Aviv (IL); Michael OTSUP, Herzliya (IL); Yonatan MEIR, Petah Tikva (IL)

(73) Assignee: InnerEye Ltd., Herzeliya (IL)

(21) Appl. No.: 18/023,059

(22) PCT Filed: Aug. 25, 2021
(86) PCT No.: PCT/IL2021/051046

§ 371 Date: Feb. 24, 2023

Related U.S. Application Data
(60) Provisional application No. 63/069,742, filed on Aug. 25, 2020.

(51) Int. Cl. A61B 5/16; A61B 5/377; A61B 5/374; A61B 5/0205; A61B 5/00; G06N 3/08

(52) U.S. Cl. CPC A61B 5/165; A61B 5/377; A61B 5/374; A61B 5/0205; A61B 5/7267; G06N 3/08

(57) ABSTRACT

A method of estimating attention comprises: receiving encephalogram (EG) data corresponding to signals collected from a brain of a subject synchronously with stimuli applied to the subject. The EG data are segmented into segments, each corresponding to a single stimulus. The method also comprises dividing each segment of the EG data into a first time-window having a fixed beginning relative to a respective stimulus, and a second time-window having a varying beginning relative to the respective stimulus. The method also comprises processing the time-windows to determine the likelihood for a given segment to describe an attentive state of the brain.

### RELATED APPLICATION

This application claims the benefit of priority of U.S. Provisional Patent Application No. 63/069,742 filed on Aug. 25, 2020, the contents of which are incorporated herein by reference in their entirety.

### FIELD AND BACKGROUND OF THE INVENTION

The present invention, in some embodiments thereof, relates to a brain wave analysis and, more particularly, but not exclusively, system and method for quantifying attention based on such analysis. Some embodiments relate to system and method for quantifying fatigue and/or mind-wandering.

Electroencephalography, a noninvasive recording technique, is one of the commonly used systems for monitoring brain activity. In this technique, electroencephalogram (EEG) data is simultaneously collected from a multitude of channels at a high temporal resolution, yielding high dimensional data matrices for the representation of single trial brain activity. In addition to its unsurpassed temporal resolution, EEG is wearable, and more affordable than other neuroimaging techniques, and has been used for various purposes, e.g., in brain computer interface (BCI) applications, where the brain activity is decoded in response to single events (trials).

Traditional EEG classification techniques use machine-learning algorithms to classify single-trial spatio-temporal activity matrices based on statistical properties of those matrices. These methods are based on two main components: a feature extraction mechanism for effective dimensionality reduction, and a classification algorithm. Typical classifiers use a sample data to learn a mapping rule by which other test data can be classified into one of two or more categories. Classifiers can be roughly divided to linear and non-linear methods. Non-linear classifiers, such as Neural Networks, Hidden Markov Model and k-nearest neighbor, can approximate a wide range of functions, allowing discrimination of complex data structures. While non-linear classifiers have the potential to capture complex discriminative functions, their complexity can also cause overfitting and carry heavy computational demands, making them less suitable for real-time applications.

Linear classifiers, on the other hand, are less complex and are thus more robust to data overfitting. Linear classifiers perform particularly well on data that can be linearly separated. Fisher Linear discriminant (FLD), linear Support Vector Machine (SVM) and Logistic Regression (LR) are examples of linear classifiers. FLD finds a linear combination of features that maps the data of two classes onto a separable projection axis. The criterion for separation is defined as the ratio of the distance between the classes mean to the variance within the classes. SVM finds a separating hyper-plane that maximizes the margin between the two classes. LR, as its name suggests, projects the data onto a logistic function.

International publication No. WO2014/170897, the contents of which are hereby incorporated by reference, discloses a method for conduction of single trial classification of EEG signals of a human subject generated responsive to a series of images containing target images and non-target images. The method comprises: obtaining the EEG signals in a spatio-temporal representation comprising time points and respective spatial distribution of the EEG signals; classifying the time points independently, using a linear discriminant classifier, to compute spatio-temporal discriminating weights; using the spatio-temporal discriminating weights to amplify the spatio-temporal representation by the spatio-temporal discriminating weights at tempo-spatial points respectively, to create a spatially-weighted representation; using Principal Component Analysis (PCA) on a temporal domain for dimensionality reduction, separately for each spatial channel of the EEG signals, to create a PCA projection; applying the PCA projection to the spatially-weighted representation onto a first plurality of principal components, to create a temporally approximated spatially weighted representation containing for each spatial channel, PCA coefficients for the plurality of principal temporal projections; and classifying the temporally approximated spatially weighted representation, over the number of channels, using the linear discriminant classifier, to yield a binary decisions series indicative of each image of the images series as either belonging to the target image or to the non-target image.

International publication No. WO2016/193979, the contents of which are hereby incorporated by reference discloses a method of classifying an image. A computer vision procedure is applied to the image to detect therein candidate image regions suspected as being occupied by a target. An observer is presented with each candidate image region as a visual stimulus, while collecting neurophysiological signals from the observer's brain. The neurophysiological signals are processed to identify a neurophysiological event indicative of a detection of the target by the observer. An existence of the target in the image is determined based on the identification of the neurophysiological event.

International publication No. WO2018/116248 discloses a technique for training an image classification neural network. An observer is presented with images as a visual stimulus and neurophysiological signals are collected from his or hers brain. The signals are processed to identify a neurophysiological event indicative of a detection of a target by the observer in an image, and the image classification neural network is trained to identify the target in the image based on such identification.

### SUMMARY OF THE INVENTION

According to an aspect of some embodiments of the present invention there is provided a method of estimating attention. The method comprises: receiving encephalogram (EG) data corresponding to signals collected from a brain of a subject synchronously with stimuli applied to the subject, the EG data being segmented into a plurality of segments, each corresponding to a single stimulus; dividing each segment into a first time-window having a fixed beginning, and a second time-window having a varying beginning, the fixed and the varying beginnings being relative to a respective stimulus; and processing the time-windows to determine the likelihood for a given segment to describe an attentive state of the brain.

According to some embodiments of the invention the varying beginning is a random beginning.

According to some embodiments of the invention the method comprises receiving additional EG data collected from a brain of a subject while deliberately being inattentive for a portion of the stimuli. The additional EG data are also segmented into a plurality of segments, each corresponding to a single stimulus. According to some embodiments of the invention the method comprises processing the segments of the additional EG data to determine an additional likelihood for a given segment to describe an attentive state of the brain; and combining the likelihood and the additional likelihood.

According to some embodiments of the invention the method comprises representing each segment of the additional EG data as a time-domain data matrix, wherein the processing comprises processing the time-domain data matrix.

According to some embodiments of the invention the method comprises representing each segment of the additional EG data as a frequency-domain data matrix, wherein the processing comprises processing the frequency-domain data matrix.

According to some embodiments of the invention the method comprises representing each segment of the additional EG data as a time-domain data matrix and as a frequency-domain data matrix, wherein the processing comprises separately processing the data matrices to provide two separate scores describing the additional likelihood, and wherein the combining comprises combining a score describing the likelihood with the two separate scores describing the additional likelihood.

According to some embodiments of the invention the method comprises receiving additional physiological data, and processing the additional physiological data, wherein the likelihood is based also on the processed additional physiological data.

According to some embodiments of the invention the additional physiological data pertain to at least one physiological parameter selected from the group consisting of amount and time-distribution of eye blinks, duration of eye blinks, pupil size, muscle activity, movement, and heart rate.

According to some embodiments of the invention the method comprises extracting spatio-temporal-frequency features from the segments, and clustering the features into clusters of different awareness states.

According to some embodiments of the invention the awareness states comprise at least one awareness state selected from the group consisting of a fatigue state, an attention state, an inattention state, a mind wandering state, a mind blanking state, a wakefulness state, and a sleepiness state.

According to some embodiments of the invention the first time-window has a fixed width. According to some embodiments of the invention the second time-window has a fixed width. According to some embodiments of the invention each of the first and the second time-windows has an identical fixed width.

According to some embodiments of the invention the second time-window has a varying width.

According to some embodiments of the invention the processing comprises applying a linear classifier. According to some embodiments of the invention the linear classifier comprises a machine learning procedure.

According to some embodiments of the invention the processing comprises applying a non-linear classifier. According to some embodiments of the invention the non-linear classifier comprises a machine learning procedure.

According to an aspect of some embodiments of the present invention there is provided a method of estimating attention. The method comprises: receiving EG data corresponding to signals collected from a brain of a subject synchronously with stimuli applied to the subject, the EG data being segmented into a plurality of segments, each corresponding to a single stimulus. The method also comprises accessing a computer readable medium storing a set of machine learning procedures, each being trained for estimating attention specifically for the subject, and being associated with a parameter indicative of a performance of the procedure. The method also comprises, for each machine learning procedure of the set, feeding the procedure with the plurality of segments, and receiving from the procedure, for each segment, a score indicative of a likelihood for the segment to describe an attentive state of the brain, thereby providing, for each segment, a set of score. The method also comprises combining the scores based on the parameters indicative of the performances, to provide a combined score; and generating an output pertaining to the combined score.

According to an aspect of some embodiments of the present invention there is provided a method of determining a task-specific attention. The method comprises: receiving EG data corresponding to signals collected from a brain of a subject engaged in a brain activity over a time period, the time period comprises intervals at which the subject performs a task-of-interest and intervals at which the subject performs background tasks; segmenting the EG data into partially overlapping segments, according to a predetermined segmentation protocol independent of the activity of the subject; assigning each segment with a vector of values, wherein one of the values identifies a type of task corresponding to an interval overlapped with the segment, and other values of the vector are features which are extracted from the segment; feeding a first machine learning procedure with vectors assigned to the segments, to train the first procedure to determine a likelihood for a segment to correspond to an interval at which the subject is performing the task-of-interest; and storing the first trained procedure in a computer-readable medium.

According to some embodiments of the invention at least one value of the vector is a frequency-domain feature.

According to some embodiments of the invention the first machine learning procedure is a logistic regression procedure.

According to some embodiments of the invention the EG data is arranged over M channels, each corresponding to a signal generated by one EG sensor, and wherein the vector comprises at least 10M features, or at least 20M features, or at least 40M features, or at least 80M features.

According to some embodiments of the invention the task-of-interest is selected from a first group consisting of tasks comprises a visual processing task, an auditory processing task, a working memory task, a long term memory task, a language processing task, and any combination thereof.

According to some embodiments of the invention the task-of-interest is one member of the first group, and the background tasks comprise all other members of the first group.

According to some embodiments of the invention the method comprises calculating a Fourier transform for each segment, and feeding a second machine learning procedure with Fourier transform to train the second procedure to determine a likelihood for a segment to correspond to an interval at which the subject is concentrated.

According to an aspect of some embodiments of the present invention there is provided a method of determining mind-wandering or inattentive brain state. The method comprises: receiving EG data corresponding to signals collected from a brain of a subject engaged in a brain activity over a time period, the time period comprises intervals at which the subject performs a no-go task. The method also comprises segmenting the EG data into segments, each being encompassed by a time interval which is devoid of any onset of the no-go task; and assigning each of the segments with a label according to a success or a failure of the no-go task in response to an onset immediately following the segment. The method also comprises training a machine learning procedure using the segments and the labels to estimate a likelihood for a segment to correspond to a time-window at which the brain is in a mind wandering or inattentive state; and storing the trained procedure in a computer-readable medium.

According to an aspect of some embodiments of the present invention there is provided a method of determining awareness state. The method comprises: receiving EG data corresponding to signals collected from a brain of a subject engaged in a brain activity over a time period; segmenting the EG data into segments according to a predetermined protocol independent of the activity of the subject; extracting classification features from the segments, and clustering the features into clusters; ranking the clusters according to an awareness state of the subject.

According to an aspect of some embodiments of the present invention there is provided a method of determining awareness state of a particular subject within a group of subjects. The method comprises: for each subject of the group receiving EG data, extracting classification features from the data, and clustering the features into a set of L clusters, each being characterized by a central vector of features, thereby providing a plurality of L-sets of central vectors, one L-set for each subject. The method also comprises clustering the central vectors into a L clusters of central vectors; and, for at least the particular subject, re-clustering the classification features, using centers of the L clusters of central vectors as initializing cluster seeds, and ranking the clusters according to an awareness state of the subject.

According to some embodiments of the invention the method comprises supplementing the classification features by the centers of the L clusters of central vectors, prior to the re-clustering.

According to some embodiments of the invention the method comprises segmenting the EG data into segments according to a predetermined protocol independent of the activity of the subject.

According to some embodiments of the invention the predetermined protocol comprises a sliding window.

According to some embodiments of the invention the predetermined protocol comprises segmentation based only on the EG data.

According to some embodiments of the invention the segmentation is according to energy bursts within the EG data.

According to some embodiments of the invention the segmentation is adaptive. For example, different segments can have different widths.

According to some embodiments of the invention the ranking is based on membership level of segments of the EG data to the clusters.

According to some embodiments of the invention the awareness states comprise at least one awareness state selected from the group consisting of a fatigue state, an attention state, an inattention state, a mind wandering state, a mind blanking state, a wakefulness state, and a sleepiness state.

According to an aspect of some embodiments of the present invention there is provided a computer software product, comprises a computer-readable medium in which program instructions are stored, which instructions, when read by a data processor, cause the data processor to execute the method as delineated above and optionally and preferably as further detailed below.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of a method suitable for estimating attention, according to some embodiments of the present invention;

FIG. 2 is a flowchart diagram of a method suitable for estimating attention, in embodiments of the invention in which the method uses labeled encephalogram (EG) data;

FIGS. 3A and 3B is a schematic illustration of an architecture of a convolutional neural network (CNN) used in experiments performed according to some embodiments of the present invention;

FIG. 4 shows trialness scores that measure the ability of a subject to be successful in a single trial, as obtained in experiments performed according to some embodiments of the present invention;

FIG. 5 shows a comparison between accuracies of a linear classifier and a CNN, as obtained in experiments performed according to some embodiments of the present invention;

FIG. 6 is a graph prepared in experiments performed according to some embodiments of the present invention to demonstrate increase in performance accuracy with data accumulation;

FIG. 7 shows normalized trialness scores, averaged across subjects, before (t<0) and after (t>0) a break (t=0), obtained in experiments performed according to some embodiments of the present invention;

FIG. 8 shows a comparison between different scores obtained in experiments performed according to some embodiments of the present invention;

FIG. 9 shows performances for detecting attentive states using four classification methods employed in experiments performed according to some embodiments of the present invention;

FIG. 10 shows an attention index, which is defined as a score obtained for each subject using the classifier that provided the highest performance for this subject, averaged over several subjects, as obtained in experiments performed according to some embodiments of the present invention;

FIGS. 11A-D show Evoked Response Potential (ERP) for four subjects, as obtained in experiments performed according to some embodiments of the present invention;

FIG. 12 shows performance of a trialness classifier, as obtained in experiments performed according to some embodiments of the present invention;

FIG. 13 shows features found to be influential on a logistic regression function employed during experiments performed according to some embodiments of the present invention;

FIGS. 14A and 14B show performances of task-specific attention classifiers, employed during experiments performed according to some embodiments of the present invention;

FIG. 15 shows performances of a concentration classifier, employed during experiments performed according to some embodiments of the present invention;

FIG. 16 is a schematic illustration of a clustering procedure, according to some embodiments of the present invention;

FIG. 17 shows cluster membership levels of data segments for a cluster associated with energy in the alpha band, as obtained in experiments performed according to some embodiments of the present invention;

FIG. 18 is a schematic illustration of a graphical user interface (GUI) suitable for presenting an output of a clustering procedure, according to some embodiments of the present invention;

FIG. 19 shows performances of a fatigue classifier employed during experiments performed according to some embodiments of the present invention;

FIG. 20 shows a mind wandering signal obtained in experiments performed according to some embodiments of the present invention;

FIG. 21 shows a performance of a mind wandering classifier employed in experiments performed according to some embodiments of the present invention;

FIGS. 22A and 22B show exemplary combined outputs for estimation of brain states, according to some embodiments of the present invention;

FIG. 23 is a flowchart diagram describing a method suitable for determining a task-specific attention and/or concentration, according to some embodiments of the present invention;

FIGS. 24A and 24B are flowchart diagrams describing methods suitable for estimating awareness state of a brain, according to some embodiments of the present invention; and

FIG. 25 is a flowchart diagram describing a method suitable for determining mind-wandering or inattentive brain state, according to some embodiments of the present invention.

### DESCRIPTION OF SPECIFIC EMBODIMENTS OF THE INVENTION

The present invention, in some embodiments thereof, relates to a brain wave analysis and, more particularly, but not exclusively, system and method for quantifying attention based on such analysis. Some embodiments relate to system and method for quantifying fatigue and/or mind-wandering.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Human observers engaged in a large number of tasks at a relatively high tasking rate (for example, as X-Ray screeners in airports that are repeatedly presented with images), oftentimes experience reduction in their level attention to the tasks they are instructed to perform, either instantaneously or over some time interval. Such a reduction may be a result of, e.g., drowsiness, mind-wandering, distractions or the like. Events at which the level of attention is reduced can be overt or covert. Overt events are those attention reduction events that are detectable by monitoring external organs of the subject. For example, when the tasks include viewing images on a screen, overt attention reduction occurs when the subject no longer looks at the screen, and can thus be detected by monitoring the subject's gaze or head direction.

Covert events are those attention reduction events in which the external organs of the subject appear to be in the same state as when the attention level was high, and so cannot be detected by monitoring the external organs. For example, when the tasks include viewing images on a screen, covert attention reduction occurs when the subject is still gazing at the screen, but his brain is in a state that does not provide adequate attention to the images on the screen.

The Inventors discovered a technique that can estimate the attention by analyzing encephalogram (EG) data. The technique can be used for detecting covert attention reduction events, and optionally and preferably also overt attention reduction events.

At least part of the operations described herein can be can be implemented by a data processing system, e.g., a dedicated circuitry or a general purpose computer, configured for receiving data and executing the operations described below. At least part of the operations can be implemented by a cloud-computing facility at a remote location.

Computer programs implementing the method of the present embodiments can commonly be distributed to users by a communication network or on a distribution medium such as, but not limited to, a floppy disk, a CD-ROM, a flash memory device and a portable hard drive. From the communication network or distribution medium, the computer programs can be copied to a hard disk or a similar intermediate storage medium. The computer programs can be run by loading the code instructions either from their distribution medium or their intermediate storage medium into the execution memory of the computer, configuring the computer to act in accordance with the method of this invention. All these operations are well-known to those skilled in the art of computer systems.

Processing operations described herein may be performed by means of processer circuit, such as a DSP, microcontroller, FPGA, ASIC, etc., or any other conventional and/or dedicated computing system.

The method of the present embodiments can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

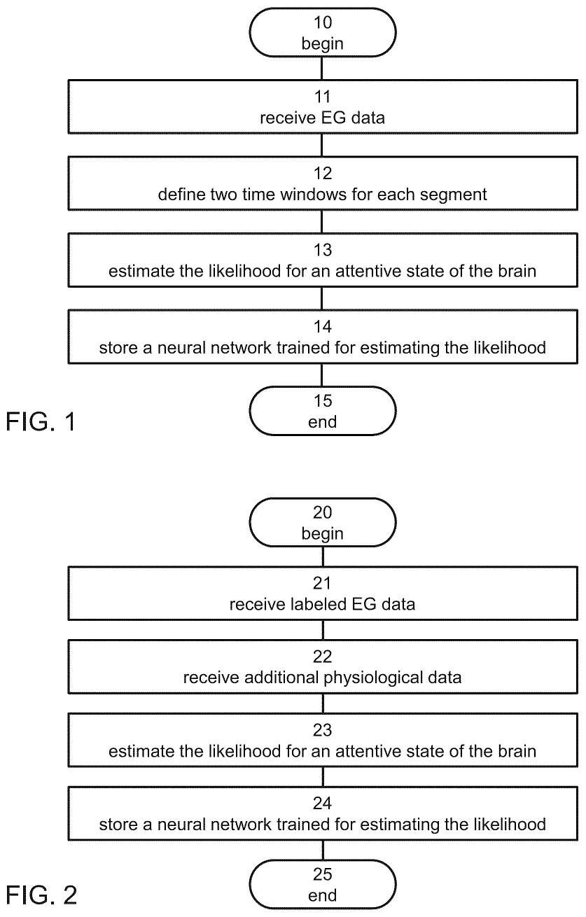

Referring now to the drawings, FIG. 1 is a flowchart diagram of the method according to various exemplary embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

The method begins at 10 and optionally and preferably continues to 11 at which encephalogram (EG) data are received. The EG data can be EEG data or magnetoencephalogram (MEG) data.

The EG data are digitized form of EG signals that are collected, optionally and preferably simultaneously, from a multiplicity of sensors (e.g., at least 4 or at least 16 or at least 32 or at least 64 sensors), and optionally and preferably at a sufficiently high temporal resolution. The sensors can be electrodes in the case of EEG, and superconducting quantum interference devices (SQUIDs) in the case of MEG.

In some embodiments of the present invention signals are sampled at a sampling rate of at least 150 Hz or at least 200 Hz or at least 250 Hz, e.g., about 256 Hz. Optionally, a low-pass filter of is employed to prevent aliasing of high frequencies. A typical cutoff frequency for the low pass filter is, without limitation, about 100 Hz.

When the neurophysiological signals are EEG signals, one or more of the following frequency bands can be defined: delta band (typically from about 1 Hz to about 4 Hz), theta band (typically from about 3 to about 8 Hz), alpha band (typically from about 7 to about 13 Hz), low beta band (typically from about 12 to about 18 Hz), beta band (typically from about 17 to about 23 Hz), and high beta band (typically from about 22 to about 30 Hz). Higher frequency bands, such as, but not limited to, gamma band (typically from about 30 to about 80 Hz), are also contemplated.

The EG data correspond to signals collected from the brain of a particular subject synchronously with stimuli applied to the subject. When a stimulus is presented to an individual, for example, during a task in which the individual is asked to identify the stimulus, a neural response is elicited in the individual's brain. The stimulus can be of any type, including, without limitation, a visual stimulus (e.g., by displaying an image), an auditory stimulus (e.g., by generating a sound), a tactile stimulus (e.g., by physically touching the individual or varying a temperature to which the individual is exposed), an olfactory stimulus (e.g., by generating odor), or a gustatory stimulus (e.g., by providing the subject with an edible substance). When the attention to the stimulus is low the response is modified, so by measuring neural activity it is possible to assess how much a person is engaged in the task.

The signals can be collected by the method, or the method can receive the previously recorded data. For example, the method can use data collected during a training session in which the particular subject was involved. The EG data are optionally and preferably segmented into a plurality of multi-channel segments, each corresponding to a single stimulus applied to the subject. For example, the data can be segmented to trials, where each multi-channel segment contains N time-points collected over M spatial channels, where each channel correspond to a signal provided by one of the sensors. The trials are typically segmented from a predetermined time (e.g., 300 ms, 200 ms, 100 ms, 50 ms) before the onset of the stimulus, to a predetermined time (e.g., 500 ms, 600 ms, 700 ms, 800 ms, 900 ms, 1000 ms, 1100 ms, 1200 ms) after the onset of the stimulus.

The method continues to 12 at which two time windows are defined for each segment. A first time-window has a fixed beginning relative to a respective stimulus, and a second time-window has a varying (e.g., random) beginning relative to the respective stimulus. The first time-window preferably begins before the onset of the stimulus and ends after the onset of the stimulus. It is therefore referred to herein as a “true” trial, because it encompasses the onset of the stimulus, and therefore contains data that correlates with the brain's response to the stimulus. The second time window has a beginning that varies among the segments, and does not necessarily encompass the onset of the stimulus. The second time window is therefore referred to herein a “sham” trial since it contains data that may or may not correlate with the brain's response to the stimulus.

The first time window is preferably fixed both with respect to the beginning and with respect to the width of the time window. The second time-window varies with respect to the beginning of the time window, but in various exemplary embodiments of the invention has a fixed width. In some embodiments of the present invention the widths of the two windows are the same or approximately the same.

Representative examples of width for the first and second time windows include, without limitation, about 10% or about 20% or about 30% or about 40% of the length of the segment. In some embodiments of the present invention the widths of the fixed and varying time windows is Δt, where Δt is about 100 ms, or about 125 ms, or about 150 ms, or about 175 ms, or about 200 ms, or about 225 ms, or about 250 ms, or about 275 ms, or about 300 ms, or about 325 ms, or about 350 ms, or about 375 ms, or about 400 ms. In some embodiments of the present invention the beginning of the fixed time window is ti ms before the onset of the stimulus, where ti is about 200, or about 175, or about 150, or about 125, or about 100, or about 75, or about 50.

The method optionally and preferably proceeds to 13 at which the time-windows defined at 12 are processed to determine the likelihood for a given segment to describe an attentive state of the brain.

The processing is preferably automatic and can be based on supervised or unsupervised learning of the data windows. Learning techniques that are useful for determining the attentive state include, without limitation, Common Spatial Patterns (CSP), autoregressive models (AR) and Principal Component Analysis (PCA). CSP extracts spatial weights to discriminate between two classes, by maximizing the variance of one class while minimizing the variance of the second class. AR instead focuses on temporal, rather than spatial, correlations in a signal that may contain discriminative information. Discriminative AR coefficients can be selected using a linear classifier.

PCA is particularly useful for unsupervised learning. PCA maps the data onto a new, typically uncorrelated space, where the axes are ordered by the variance of the projected data samples along the axes, and only axes that reflect most of the variance are maintained. The result is a new representation of the data that retains maximal information about the original data yet provides effective dimensionality reduction.

Another method useful for identifying a target detection event employs spatial Independent Component Analysis (ICA) to extract a set of spatial weights and obtain maximally independent spatial-temporal sources. A parallel ICA stage is performed in the frequency domain to learn spectral weights for independent time-frequency components. PCA can be used separately on the spatial and spectral sources to reduce the dimensionality of the data. Each feature set can be classified separately using Fisher Linear Discriminants (FLD) and can then optionally and preferably be combined using naive Bayes fusion, by multiplication of posterior probabilities).

In various exemplary embodiments of the invention the method employs a Spatially Weighted Fisher Linear Discriminant (SWFLD) classifier to the data windows. This classifier can be obtained by executing at least some of the following operations. Time points can be classified independently to compute a spatiotemporal matrix of discriminating weights. This matrix can then be used for amplifying the original spatiotemporal matrix by the discriminating weights at each spatiotemporal point, thereby providing a spatially-weighted matrix.

Preferably the SWFLD is supplemented by PCA. In these embodiments, PCA is optionally and preferably applied on the temporal domain, separately and independently for each spatial channel. This represents the time series data as a linear combination of components. PCA is optionally and preferably also applied independently on each row vector of the spatially weighted matrix. These two separate applications of PCA provide a projection matrix, which can be used to reduce the dimensions of each channel, thereby providing a data matrix of reduced dimensionality.

The rows of this matrix of reduced dimensionality can then be concatenated to provide a feature representation vector, representing the temporally approximated, spatially weighted activity of the signal. An FLD classifier can then be trained on the feature vectors to classify the spatiotemporal matrices into one of two classes. In the present embodiments, one class corresponds to a true trial, and another class corresponds to a sham trial.

In some embodiments of the present invention a nonlinear procedure is employed. In these embodiments the procedure can include an artificial neural network. Artificial neural networks are a class of machine learning procedures based on a concept of inter-connected computer program objects referred to as neurons. In a typical artificial neural network, neurons contain data values, each of which affects the value of a connected neuron according to a pre-defined weight (also referred to as the “connection strength”), and whether the sum of connections to each particular neuron meets a pre-defined threshold. By determining proper connection strengths and threshold values (a process also referred to as training), an artificial neural network can achieve efficient recognition of patterns in data. Oftentimes, these neurons are grouped into layers. Each layer of the network may have differing numbers of neurons, and these may or may not be related to particular qualities of the input data. An artificial neural network having an architecture of multiple layer belongs to a class of artificial neural networks referred to as deep neural network.

In one implementation, called a fully-connected network, each of the neurons in a particular layer is connected to and provides input values to each of the neurons in the next layer. These input values are then summed and this sum is used as an input for an activation function (such as, but not limited to, ReLU or Sigmoid). The output of the activation function is then used as an input for the next layer of neurons. This computation continues through the various layers of the neural network, until it reaches a final layer. At this point, the output of the fully-connected network can be read from the values in the final layer.

Convolutional neural networks (CNNs) include one or more convolutional layers in which the transformation of a neuron value for the subsequent layer is generated by a convolution operation. The convolution operation includes applying a convolutional kernel (also referred to in the literature as a filter) multiple times, each time to a different patch of neurons within the layer. The kernel typically slides across the layer until all patch combinations are visited by the kernel. The output provided by the application of the kernel is referred to as an activation map of the layer. Some convolutional layers are associated with more than one kernel. In these cases, each kernel is applied separately, and the convolutional layer is said to provide a stack of activation maps, one activation map for each kernel. Such a stack is oftentimes described mathematically as an object having D+1 dimensions, where D is the number of lateral dimensions of each of the activation maps. The additional dimension is oftentimes referred to as the depth of the convolutional layer.

In some embodiments of the present invention the artificial neural network employed by the method is a deep learning neural network, more preferably a CNN.

The artificial neural network can be trained according to some embodiments of the present invention by feeding an artificial neural network training program with labeled window data. For example, each window can be represented as a spatiotemporal matrix having N columns and M rows (or vise versa), wherein each matrix element stores a value representing the EG signal sensed by a particular EG sensor at a particular time point within the window. Each window that is fed to the training program is labeled. In some embodiments of the present invention a binary labeling is employed during the training. For example, a window can be labeled as being of the fixed-beginning first window type (corresponding to a true trial) or of the varying-beginning second window type (corresponding to a sham trial). Since for each segment, in principle, two types of windows can be defined, the number of labeled windows that are fed to the artificial neural network training program can be is twice the number of segments in the data, thus improving the classification accuracy of the training process.

The training process adjusts the parameters of the artificial neural network, for example, the weights, the convolutional kernels, and the like so as to produce an output that classifies each window as close as possible to its label. The final result of the training is a trained artificial neural network with adjusted weights assigned to each component (neuron, layer, kernel, etc.) of the network. The trained artificial neural network can then be stored 14 in a computer readable medium, and can be later used without the need to re-train it. For example, once pulled from computer readable medium, the trained artificial neural network can receive an un-labeled EG data segment and produce a score, typically in the range [0, 1], which estimates the likelihood that the segment describes an attentive state of the brain. Unlike the artificial neural network training program that is fed with a first and a second time-window for each segment of the EG data, the subsequently used trained artificial neural network need not be fed by two time-windows per segment. Rather, the trained artificial neural network can be fed by the EG data segments themselves, optionally and preferably following some preprocessing operations such as, but not limited to, filtering and removal or artifacts.

A representative example of an architecture of a CNN suitable for the present embodiments is provided in the Examples section that follow.

Method 10 ends at 15.

FIG. 2 is a flowchart diagram of the method in embodiments of the invention in which the method uses labeled EG data. In these embodiments, the method begins at 20 and continues to 21 at which the method receives EG data collected from the subject's brain while the subject is requested to be deliberately inattentive for a portion of the applied stimuli. As for the data received at 11 (FIG. 1) the EG data received at 21 are also segmented into multi-channel segments, each corresponding to a single stimulus. Unlike the data received at 11, the segments of the EG data received at 21 are labeled according to the deliberate attention level of the subject. Specifically, each segment of these EG data is optionally and preferably labeled using a binary label indicative of whether or not the subject was deliberately inattentive during the time interval that is encompassed by the respective segment. The EG data received at 21 are thus referred to as labeled EG data.

In some embodiments of the present invention the method continues to 22 at which additional physiological data are received. The additional physiological data can include any type of data that can be correlated with the attention. For example, such data can include data that is indicative of occurrences of overt attention reduction events. Representative examples of additional physiological data suitable for the present embodiments include, without limitation, data pertaining to a physiological parameter selected from the group consisting of amount of eye blinks, duration of eye blinks, pupil size, muscle activity, movement, and heart rate.

The method can proceed to 23 at which at which the segments of the labeled EG data are processed to determine the likelihood for a given segment to describe an attentive state of the brain. The processing 23 is preferably automatic and can be based on any of the aforementioned supervised or unsupervised learning techniques, except that in method 20 the segments are labeled according to the deliberate attentive state of the subject, rather than according to the type of the window that has been defined.

Preferably, the processing 23 is by an artificial neural network as further detailed hereinabove. Since each segment is assigned with one label (e.g., “0” for attentive state, or “1” for inattentive state), the number of labeled segments that are fed to the artificial neural network training program in method 20 is the same or less the total number of segments in the data received at 21. In embodiments of the present invention in which additional physiological data are received at 22, the additional physiological data are also fed into the artificial neural network training program. Preferably values of the additional physiological data are associated with the respective window, based on the time point at which they were recorded. The additional physiological data serve as additional labels to the segments and therefore improve the accuracy of the classification. For example, when the additional physiological data relate to eye blinks, existence of long eye blinks or many short eye blinks may indicate that the brain is likely to be in inattentive state, and the respective label can be labeled as such.

In method 10 above, the input to the artificial neural network training program included the windows defined at 12. As such, the input is in the time domain, for example, using the aforementioned spatiotemporal matrix. In method 20, it is not necessary for the input to be in the time domain, since it is not based on time windows that have been defined for each segment. Thus, in some embodiments of the present invention the input to the artificial neural network training program is arranged in the time domain, and in some embodiments of the present invention the input to the artificial neural network training program is arranged in the frequency domain. Also contemplated, are embodiments in which two artificial neural network are trained: a time-domain artificial neural network is trained by feeding the artificial neural network training program with data arranged in the time domain, and a frequency-domain artificial neural network is trained by feeding the artificial neural network training program with data arranged in the frequency domain.

In the time domain, the input data can be arranged according to the principles described with respect to method 10 above. In the frequency domain, the input data can be arranged by applying a Fourier transform to each of the multi-channel segments producing a spatiospectral matrix wherein each matrix element stores a value representing the EG signal sensed by a particular EG sensor at a particular frequency bin. A typical number of frequency bins is from about 10 to about 100 bins over a frequency range of from about 1 Hz to about 30 Hz. Thus, both the time-domain and frequency-domain artificial neural networks are trained to score each segment according to the likelihood that the brain is in attentive state during the time interval encompassed by the segment. The difference between these networks is that the input to the time-domain network is based on time bins, the input to the frequency-domain artificial network is based on frequency bins.

The trained artificial neural network(s) can then be stored 24 in a computer readable medium, and can be later used without the need to re-train them, as further detailed hereinabove. Method 20 ends at 25.

The Inventors found that while both method 10 and method 20 provide a likelihood for the attentive state of the brain, the interpretation of the produced likelihood (e.g., of the output of the trained artificial neural network) is not the same.

Method 10 determines the likelihood based on a statistical observation that a time window which does not correlate with the stimulus can be used to classify the state of the brain with respect to the task the subject is requested to perform. Thus, the likelihood provided by method 10 assesses the similarity between a given trial and a trial at which the subject successfully performed the task. In a sense, the likelihood provided by method 10 is a measure of the ability of the subject to be successful in a single trial. The Inventors term this measure as “trialness,” and the artificial neural network trained using method 10 is referred to as the trialness network.

Method 20 determines the likelihood based on ground truth labels and therefore provide the likelihood that the reason that the subject was unable to successfully perform the task is inattention, and not, for example, some other reason.

The scores provided by the artificial networks trained using methods 10 and 20 can optionally and preferably be combined. For example, unlabeled EG data, that were collected from a brain of a specific subject synchronously with stimuli applied to the subject over a time period, can be segmented into a set of segments, where each segment corresponds to a single stimulus. A given unlabeled segment can be fed into each of the trained networks. Each of these network produces a score for the given unlabeled segment, thus providing a set of scores for the given unlabeled segment, one score for each network. The set of scores can then be combined to provide a combined score that describes the attention state of the specific subject during the time interval that overlaps with the given unlabeled segment.

Preferably, the combination of the scores is based on performance characteristics of the trained artificial neural networks for the specific subject. Thus, in various exemplary embodiments of the invention each trained artificial network is subjected to a validation process at which its performance characteristics are determined. This can be done following the training of the artificial neural network. Typically, the data available before the network is trained is divided into a training dataset that is fed to the training program, and a validation dataset that is fed to the trained networks in order to compare the outputs of the trained networks with the actual attention of the subject, and validate the ability of the network to predict the attention state of the subject.

The validation can in some embodiments of the present invention comprise applying statistical analysis to the outputs generated by each trained artificial neural network in response to the validation dataset. Such analysis can include computing a statistical measure, e.g., a measure that characterizes the receiver operating characteristic (ROC) curve produced by the scores of the segments. For example, the measure can be the area under the ROC curve (AUC). Other or additional statistical measures that can be computed during the validation process, and be used according to some embodiments of the present invention to combine the scores, including, without limitation, at least one statistical measure selected from the group consisting of number of true positives, number of true negatives, number of false negatives, number of false positives, sensitivity, specificity, total accuracy, positive predictive value, negative predictive value, and Mathews correlation coefficient.

In some embodiments of the present invention the performance characteristic associated with each the networks trained by methods 10 and 20 is also stored in a computer readable medium, and are pulled together with the trained networks in order to combine the scores. Additionally, or alternatively, a set of weights calculated based on the performance characteristics can be stored in a computer readable medium, and be pulled together with the trained networks in order to combine the scores.

A representative example of set of weights that can be calculated according to some embodiments of the present invention is a set {W} including weights wᵢ∈{W}, defined as the ratio (Pᵢ−P₀)/(ΣᵢPᵢ−nP₀), where Pᵢ is the performance characteristic of the ith network (e.g., the AUC of the ith network), ΣᵢPᵢ is a sum of the performance characteristics of all the networks, n is the number of networks that are used for producing the combined score (i=1, 2, . . . , n), and P₀ is a parameter that is optionally and preferably not specific to the subject. For example, for performance characteristics that are in the range [0,1], P₀ can be set to be about 0.5.

The combined score of a given unlabeled segment is optionally and preferably calculated as a weighted sum of the scores provided by each of the networks, using the ratios wᵢ as the weights for the sum. Specifically, denoting by Sᵢ the score provided by the ith network to the given unlabeled segment, the combined score S_{TOT} of the segment is S_{TOT}=w₁S₁+w₂S₂+ . . . +wₙSₙ, where n is the number of trained networks that are used for scoring the segment.

In some embodiments of the present invention a score provided by the trialness network is combined with a score provided by a time-domain artificial neural network trained using method 20, in some embodiments of the present invention a score provided by the trialness network is combined with a score provided by a frequency-domain artificial neural network trained using method 20, in some embodiments of the present invention a score provided by a time-domain artificial neural network trained using method 20, is combined with a score provided by a frequency-domain artificial neural network trained using method 20, and in some embodiments of the present invention a score provided by the trialness network is combined with a score provided by a time-domain artificial neural network trained using method 20 and with score provided by a frequency-domain artificial neural network trained using method 20.

The inventors of the present invention discovered that EG data can also be used for estimating the attention of a subject in cases in which the EG data are not synchronized with stimuli. This is advantageous because it allows estimating the likelihood that a subject's brain is in an attentive state while the subject performs tasks that are not driven by stimuli. For example, the subject can perform a task randomly, or within time intervals selected by the subject himself or herself. The technique is useful for cases in which it is desired to estimate the likelihood that the subject is attentive to a specific task-of-interest, or to cases in which it is desired to estimate the likelihood that the subject is concentrated in a non-specific task. The technique of the present embodiments is also useful in cases in which it is desired to estimate the likelihood that the brain of the subject is in a fatigue or a mind wandering state.

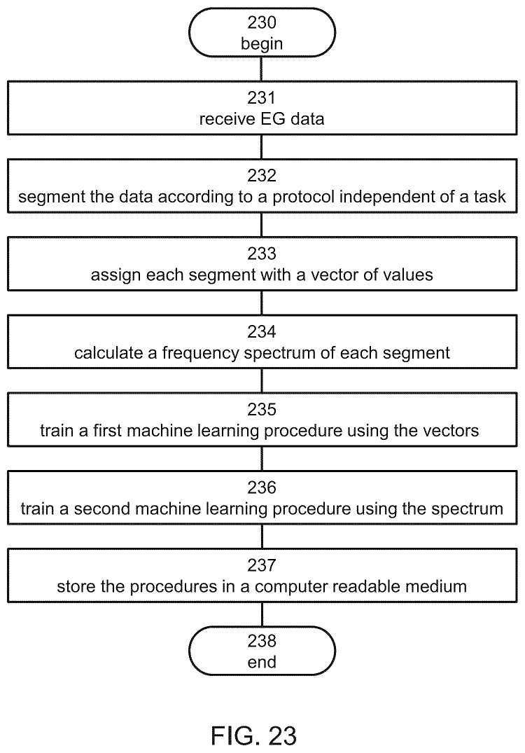

FIG. 23 is a flowchart diagram describing a method suitable for determining a task-specific attention and/or concentration, according to some embodiments of the present invention. The method begins at 230 and continues to 231 at which EG data are received as further detailed hereinabove. The EG data correspond to signals collected from the brain of a subject engaged in a brain activity. During the brain activity there are optionally and preferably intervals at which the subject performs the task-of-interest and intervals at which the subject performs background tasks. The task-of-interest can be, for example, a task selected from the group consisting of a visual processing task, an auditory processing task, a working memory task, a long term memory task, a language processing task, and a combination of two or more of these tasks. The background tasks can also be selected from the same group of tasks, with the provision that they do not include the task-of-interest itself.

The method optionally and preferably continues to 232 at which the EG data are segmented into segments, preferably, partially overlapping segments. In some embodiments of the present invention segmentation is according to a predetermined segmentation protocol that is independent of the activity of the subject.

The protocol is independent of the activity of the subject in the sense that no signal that induces the subject's activity is used to trigger the beginning or end of the segment or to otherwise define the segment. This is unlike segmentation in a conventional Evoked Response Potential trial in which a segmentation procedure locks on signals that are used to generate or transmit stimuli to the subject.

A representative example of a segmentation protocol that is independent of the activity of the subject and that is suitable for the present embodiments, include, without limitation, use of a sliding window of predetermined width (or predetermined set of widths) and predetermined overlap (or predetermined set of overlaps). Also contemplated, are embodiments in which the segmentation protocol is based only on the EG data. For example, segments can be defined when the EG data or a property thereof satisfy some predetermined criterion (e.g., exceed some threshold, falls within a range of thresholds, or the like).

The method can proceed to 233 at which a vector is assigned to each segment. One of the components of the vector identifies a type of the task (either the task-of-interest or one of the background tasks) that corresponds to a time interval that is overlapped with the segment, and other components of the vector are features which are extracted from the segment. For example, one component of the vector can be a label indicative that the task performed by the subject during the respective time interval is the task-of-interest, and other components can be extracted features. Another example is a vector in which one component is a label indicative that the task performed by the subject during the respective time interval is one of the background tasks, and the other components are extracted features.

The extracted features can be of various types, such as, but not limited to, temporal features, frequency features, spatial features, spatiotemporal features, spatiospectral features, spatio-temporal-frequency features, statistical features, ranking features, counting features, and the like. Preferably, the number of features is larger than the number of EG channels, more preferably more than 10 times the number of EG channels, more preferably more than 20 times the number of EG channels, more preferably more than 40 times the number of EG channels, more preferably more than 80 times the number of EG channels. Representative examples of features suitable for the present embodiments are provided in the Examples section that follows (see Table 5.1).

In some embodiments of the present invention the method proceeds to 234 at which a Fourier transform is calculated for each segment, providing the frequency spectrum of the EG data within the segment. Optionally and preferably, a low pass filter is applied to the Fourier transform. The cutoff frequency of the low pass filter can be from about 40 Hz to about 50 Hz, e.g., about 45 Hz.

The method optionally and preferably proceeds to 235 at which the vectors assigned to the segments are used for training a machine learning procedure to determine a likelihood for a segment to correspond to an interval at which the subject is performing the task-of-interest. In various exemplary embodiments of the invention the training of the procedure is specific both to the subject and to the task-of-interest for which attention is to be estimated. Thus, when there is more than one subject, the training process is preferably repeated separately for each subject, producing a plurality of trained machine learning procedure. Similarly, when it is desired to determine a likelihood for a segment to correspond to an interval at which the subject is performing another specific task, the training process is preferably repeated for the other specific task, producing a separate trained machine learning procedure for each task-of-interest.

The training is specific to the subject in that the features that form the vectors are extracted from EG data describing the brain activity of the subject. The training is specific to the task-of-interest in that the component of the vector that identifies whether the task is the task-of-interest or one of the background tasks, is set based on the task that has been a priori identified as the task-of-interest.

The machine learning procedure can be any of the aforementioned types of machine learning procedures. In experiments performed by the present Inventors a machine learning procedure of the logistic regression type has been employed. In embodiments in which logistic regression procedure is employed, the training process adapts a set of coefficients that define logistic regression function so that once the function is applied to the features of the vector that correspond to a given segment, the logistic regression function returns the label component of that vector. The number of coefficients in the set is typically the same as the number of features that in the vector.

In some embodiments of the present invention the method proceeds to 236 at which the spectrum obtained at 234, optionally and preferably following the filtering, is used for training another machine learning procedure to determine a likelihood for a segment to correspond to an interval at which the subject is concentrated. The machine learning procedure trained at 236 can be any of the aforementioned types of machine learning procedures. In experiments performed by the present Inventors a CNN has been employed.

Like the training at 234, the training at 236 is specific to the subject, and so for a plurality of subject, a respective plurality of machine learning procedures are preferably trained. Unlike the training at 234, the training at 236 is not specific to the task. This can be achieved by labeling the segments non-specifically with respect to the identity of the task. Thus, according to some embodiments of the present invention the training 236 comprises labeling both segments that correspond to the task-of-interest and segments that correspond to background tasks using the same label. Segments that correspond to time intervals during which the subject is not engaged in any task (or, equivalently, being engaged in activity that represent lack of concentration), are labeled with a label that is different from the label that is assigned to the segments that correspond to tasks. The training process thus adjust the parameters of the machine learning procedure, wherein the goal of the adjustment is that when the parameters are applied to a spectrum, the output of the machine learning procedure is close, as much as possible, to the label associated with that spectrum.

When the output of the procedure trained at 236 is close to the label that is assigned to segments that correspond to a task (either the task-of-interest or a background task), the method can determine that it is likely that the subject is concentrated. Conversely, when the output of the procedure is close to the label that is assigned to segments that do not correspond to any task, the method can determine that it is likely that the subject is not concentrated. The method can set the output of the procedure as a score that defines the likelihood.

The trained machine learning procedures can then be stored 237 in a computer readable medium, and can be later used without the need to re-train them, as further detailed hereinabove.

Method 230 ends at 238.

It is appreciated that while method 230 has been described in the context of determining both a task-specific attention and concentration or lack thereof, this need not necessarily be the case, since, for some applications, it may be desired to determine a task-specific attention but not concentration, and for some applications, it may be desired to determine a concentration but not task-specific attention. In the former case (determining only task-specific attention) operations 234 and 236 can be skipped. In the latter case (determining only concentration) operations 233 and 235 can be skipped.

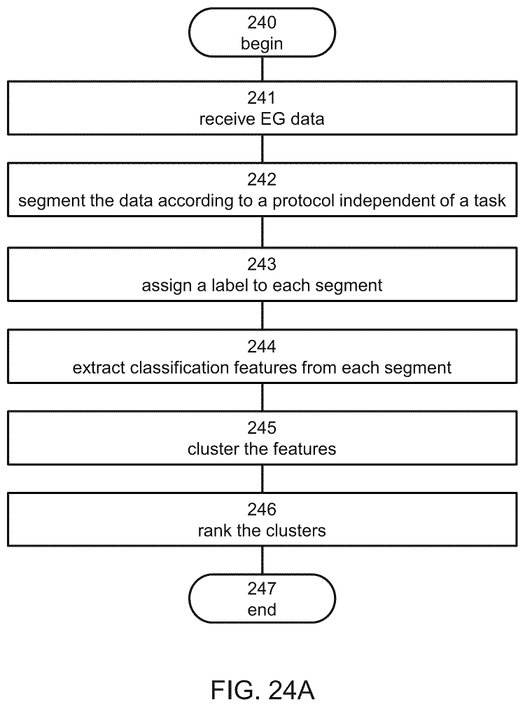

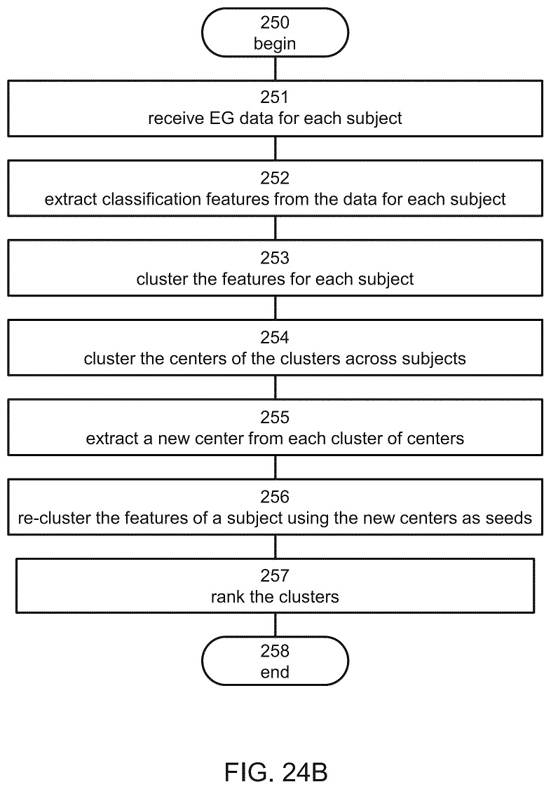

Reference is now made to FIGS. 24A and 24B which are flowchart diagrams describing methods suitable for estimating awareness state of a brain, according to some embodiments of the present invention. The flowchart diagram in FIG. 24A can be used when it is desired to determine whether the brain of a single subject is in a specific awareness state, and flowchart diagram in FIG. 24B can be used when it is desired to determine whether the brain of a particular subject within a group of subjects is in a specific awareness state. The specific awareness state can be any one of the awareness states that a brain may assume, including, without limitation, a fatigue state, an attention state, an inattention state, a mind wandering state, mind blanking state, a wakefulness state, and a sleepiness state.

Referring to FIG. 24A, the method begins at 240 and continues to 241 at which EG data are received, as further detailed hereinabove. The EG data correspond to signals collected from the brain of a subject engaged in a brain activity.

The method proceeds to 242 at which the EG data are segmented into segments, preferably according to a segmentation protocol. Preferably, the segmentation protocol is predetermined, and more preferably the segmentation protocol is predetermined and is independent of the activity of the subject, as further detailed hereinabove. In some embodiments the segmentation protocol employs a sliding window, as further detailed hereinabove, and in some embodiments the segmentation protocol is based only on the EG data, as further detailed hereinabove. Preferably, but not necessarily, the segments were defined according to energy bursts within the EG data. This can be achieved, for example, by applying Hilbert transform to each channel of the EG data to obtain an energy band envelope of the channel, and applying thresholding to the energy band envelope to identify time intervals at which the energy exceeds a predetermined threshold (energy burst). Segments can then be defined based on the identified time intervals.

The method can proceed to 243 at each of the segments is assigned with a label. The label is selected according to the task the subject is requested to perform during the time interval that overlaps with the respective segment and according to the awareness state that it is desired to estimate. In various exemplary embodiments of the invention the label is binary. As a representative example, consider a case in which it is desired to estimate the likelihood that the subject's brain is in a fatigue state. Consider further that during the time period over which the EG signals were collected, there are time intervals at which the subject is requested to perform tasks that require attention (e.g., data entry, reading, image viewing, driving, etc.), and time intervals at which the subject is requested not perform any such task and to mimic a fatigue state (e.g., by closing the eyes). In this case, the segments that overlap with the interval at which the subject perform tasks that require attention are assigned with one label (e.g., a “0”), and the segments that overlap with the interval at which the subject mimic a fatigue state are assigned with a different label (e.g., a “1”).

The method proceeds to 244 at which classification features are extracted from each segment. The classification features are optionally and preferably based at least on the frequency of the EG data in the segment. For example, the method can determine, for example, using a Fourier Transform, the brain wave bands within the segment (e.g., Alpha band, Beta band, Delta band, Theta band and Gamma band), and extract one or more features for each brain wave band. A representative example of a feature that can be extracted is the energy content of each brain wave band. These embodiments are particularly useful when the segmentation 242 employs a sliding window. When the segmentation is according to energy bursts the features can include, at least one of: peak amplitude of the burst in the respective frequency band, the area under the envelope curve in the respective frequency band, and the duration of the burst in the respective frequency band.

The number of features that are extracted for each segment is denoted D, and so at 244 each segment is assigned with a D-dimensional feature vector.

The method continues to 245 at which a clustering procedure is applied to the features extracted at 244, initializing each cluster at a seed. The present embodiments contemplate any clustering procedure, such as, but not limited to, an Unsupervised Optimal Fuzzy Clustering (UOFC) procedure. Preferably, the clustering is executed to provide a predetermined number, L, of clusters. The initial cluster seeds in the clustering procedure can be random, or, more preferably, it can be an input to the method (e.g., read from a computer readable medium). A representative example of a technique for calculating the cluster seeds is provided below.

The method optionally and preferably continues to 246 at which the clusters are ranked according to the awareness state of the subject. The ranking can be according to membership level of segments of the EG data to the clusters. Specifically for each cluster, the membership levels of all the segments that are labeled with a label that identifies the awareness state of interest can be combined (e.g., summed, averaged, etc.) to provide a ranking score for the cluster, and the cluster that yields the highest ranking score can be defined as a cluster that characterizes the awareness state of interest. With reference to the aforementioned exemplary case in which it is desired to estimate the likelihood that the subject's brain is in a fatigue state, the ranking score of each cluster can be computed by combining the membership levels of all the segments that are labeled with “1,” and the cluster that yields the highest ranking score can be defined as a cluster that characterizes a fatigue state. The membership level is optionally and preferably in the range [0,1]. The membership level can be defined to be proportional to 1/d_{i,j}, where d_{i,j}, is the distance of the jth segment features to the ith cluster. Conveniently, a membership matrix that represent the membership level of each segment to a given cluster can be constructed and used for the ranking.

The method ends at 247.

The parameters of the clusters obtained by method 240 can optionally and preferably be stored in a computer readable medium, for future use. For example, in some embodiments of the present invention the coordinates in the feature space of the centers of one or more, or each, of the clusters can be stored in the computer readable medium, for future use. Preferably, at least the coordinates of the center of the cluster that characterizes the awareness state of interest are stored.

The stored cluster parameters can be used for assigning an awareness state score to unlabeled data segments of the same subject. Such unlabeled data segments are typically obtained by collecting EG signals from the brain of the same subject during a later session, digitizing the signals to form EG data, and segmenting the data according a segmentation protocol, e.g., a protocol that is predetermined, and more preferably a protocol that is predetermined and is independent of the activity of the subject. With reference to the aforementioned exemplary case in which it is desired to estimate the likelihood that the subject's brain is in a fatigue state, the membership level of a given unlabeled data segment to a stored cluster that was previously defined as characterizing a fatigue state can be computed (e.g., by computing the distance in the feature space between the segment's feature vector and the cluster's center), and the likelihood that the brain is in a fatigue state during the time interval that overlaps with the given unlabeled data segment can be estimated based on this membership level. In embodiments of the invention in which the membership level is in the range [0,1], the likelihood can be the membership level itself. Alternatively the likelihood can be defined by normalizing the membership level.

Referring to FIG. 24B, the method begins at 250 and continues to 251 at which EG data are received, for each of the subjects in a group of subjects. The EG data correspond to signals collected from the brain of a respective subject that is engaged in a brain activity. Optionally and preferably, the EG data of each subject is segmented and labeled, as further detailed hereinabove. The method continues to 252 at which classification features are extracted from the EG data collected for each subject, as further detailed hereinabove. At 253 the features are clustered, optionally and preferably using random initialization seeds, for each subject separately. Preferably, the clustering is executed to provide a predetermined number, L, of clusters. Each of the obtained cluster is characterized by a D-dimensional central vector of features, so that operation 253 provides a plurality of L-sets of central vectors, one L-set for each subject.

Herein “L-set” means a set including L elements.

The method continues to 254 at which the D-dimensional central vectors are clustered across the group of subjects. The clustering can be using any clustering procedure, including, without limitation, a UOFC procedure. Preferably, the clustering is executed to provide the same number, L, of clusters, as at 253. Each of the clusters provided at 254 also has a center, and the method optionally and preferably extract 255 the center from each of the clusters provided by operation 254, resulting in a total of L new cluster centers. In some embodiments of the present invention the method proceeds to 256 at which the features of a particular subject of the group are re-clustered, except that the seeds for the clustering operation are the L new cluster centers provided at 255.

Optionally and preferably, prior to the re-clustering 256, the collection of classification features extracted at 252 is supplemented by the new cluster centers extracted at 255, so that the collection of classification features to which the re-clustering 256 is applied, is greater than the collection of classification features to which the clustering 253 is applied. The Inventors found that such an enlargement of the collection stabilizes the performance of the method.

At 257 the method ranks the clusters according to the awareness state of the subject, as further detailed hereinabove, and at 258 the method ends.

The parameters of one or more of the clusters obtained by method 250 can optionally and preferably be stored in a computer readable medium, for future use, as further detailed hereinabove. The stored cluster parameters can be used for assigning an awareness state score to unlabeled data segments a subject, which can be the same subject for which the clustering process was applied by method 250, or alternatively, a different subject. In other words, once the cluster parameters are stored they can be treated as universal and be used for any subject.

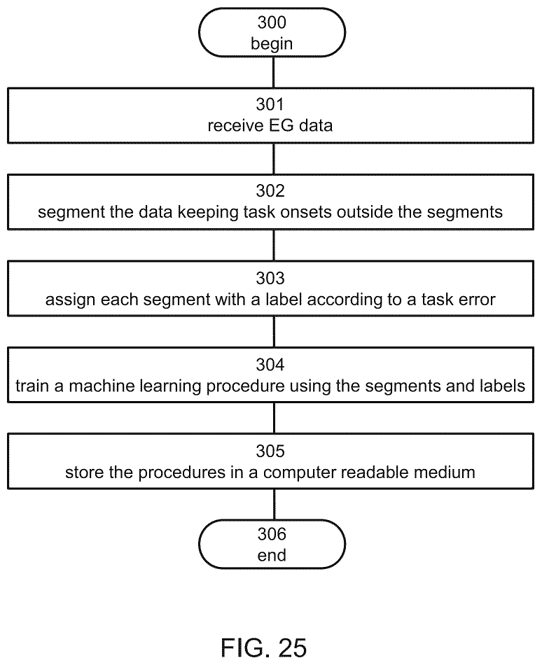

FIG. 25 is a flowchart diagram describing a method suitable for determining mind-wandering or inattentive brain state, according to some embodiments of the present invention. The method begins at 300 and continues to 301 at which EG data are received as further detailed hereinabove. The EG data correspond to signals collected from the brain of a subject engaged in a brain activity over a time period, where the time period comprising intervals at which the subject performs a no-go task.

A no-go task is a task in which the subject is requested to response to a situation unless the situation satisfies some criterion in which case the subject is requested to make no response. For example, the subject can be presented with a series of digits, and requested to respond to the currently presented digit (e.g., by typing the digit), unless the digit satisfies some criterion (e.g., the digit is “3”) in which case the subject is requested not to respond.

The method can continue to 302 at which the EG data are segmented. The segmentation is preferably such that the onsets of the no-go task (in the above example, the time instances at which the digit “3” is displayed) are all kept outside the segments. In other words, the segmentation is such that each segment is encompassed by a time interval which is devoid of any onset of the no-go task. Preferably, the end of each segment is t ms before any onset of the no-go task, wherein t is at least 50 or at least 100 or at least 150 or at least 200.

At 303 each of the segments is assigned with a label according to a commission error of the subject with respect to an onset immediately following the segment. Specifically, when the subject responds to the onset immediately following the segment (a commission error), a first label, e.g., “1”, is assigned to the segment, and when the subject makes no response to the onset immediately following the segment (a correct rejection), a second label, e.g., “0”, is assigned to the segment.

the method optionally and preferably continues to 304 at which the segments defined at 302 and the labels assigned at 304 are used to train a machine learning procedure to estimate a likelihood for a segment to correspond to a time-window at which the brain of the subject is in a mind wandering state. The Inventors found that by keeping the onsets outside the segments and analyzing the EG data with segments that are before the onset, mind wandering states can be identified, based on the labeling.

Consider for example a segment that is immediately before a commission error. Since the subject has made an error in the onset immediately after the segment, it is likely that the subject was in a mind wandering state immediately before the onset. The machine learning procedure captures the EG data patterns of all such segments and attempts to find similarities in these patterns. Consider on the other hand a segment that is immediately before a correct rejection. Since the subject has properly identified that no response should be made to the onset immediately after the segment, it is likely that the subject was not in a mind wandering state immediately before the onset. The machine learning procedure also captures and attempts to find similarities between the EG data patterns of these segments.

The trained machine learning procedures can then be stored 305 in a computer readable medium, and can be later used without the need to re-train it. At run time, an unlabeled segment is fed to the trained machine learning procedure. The procedure determines to which of the EG patterns in the training data the unlabeled segment is more similar, and accordingly issues an output.

The method ends at 306.

Two or more of methods 10, 20, 230, 240, 250 and 300 can be combined together to provide a combined method that provide a score for each of the aforementioned states. The method can be executed serially, in any order, or in parallel.

As used herein the term “about” refers to ±10% The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Estimation of “Trialness”

### Methods

EEG signals were recorded from the brain, while the subject was presented with a set of images as a visual stimulus. The EEG signals were digitized to provide EEG data, and the data were preprocessed by applying a band pass filter 1-20 Hz, and by removing artifacts. The data was segmented from −100 ms to 900 ms relative to image onset. From these trials two sets of trimmed windows were extracted. Fixed beginning windows (“true trials”) were defined from −100 ms to 175 ms (window width 275 ms) relative to image onset, and variable beginning windows (“sham trials”) were defined to include a random beginning with the same width as the true trials.

The defined windows were used for training a linear classifier as well as a nonlinear classifier (a CNN in the present example).

After training, the classifiers were fed with EEG data obtained for the same subject, but during a different image-review session. Each classifiers produced a set of trialness scores which was smoothed by moving average filter with variable window size, selected based on the required accuracy and latency. In this example, window sizes of 1-25 seconds were used.

Linear Classifier

Each input segment included N EEG data samples over M channels.

For data matrix X (data sample by channels, per segment) a weighting matrix U (channels by data samples) was created using FLD technique. The data matrix X was multiplied by the weighting matrix U to amplify differences between trials and non-trials. For data reduction to K components, a projection matrix A (samples by K by channels) was computed using temporal PCA, independently for each channel. The top K components of the PCA were kept. In this Example, K was set to be 6. FLD was computed to choose points in time, for which components and channels are weighed more heavily.

CNN Classifier

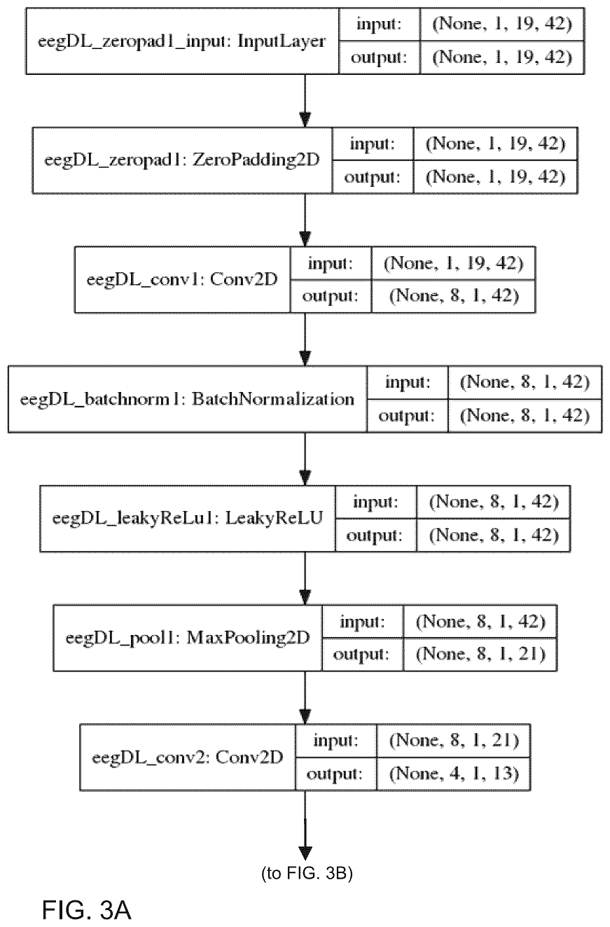

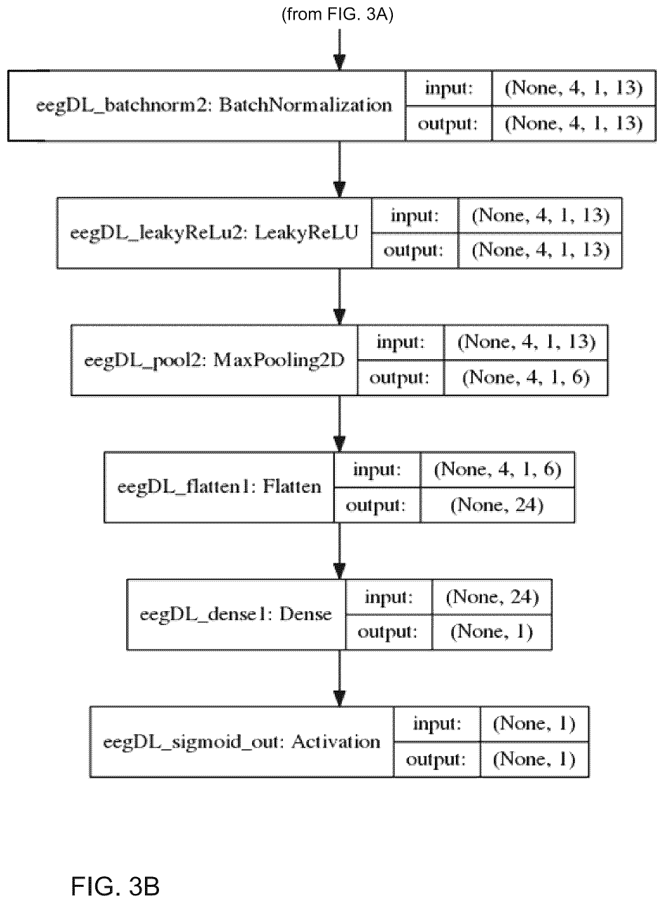

An architecture of a CNN used in the present Example for N=42 time points and M=19 channels is illustrated in FIGS. 3A-B.

Results

Single Subject

The subject performed 3 tasks: Attentive task—look for images including targets, Inattentive task—do not look at the images, and Shutting the eyes.

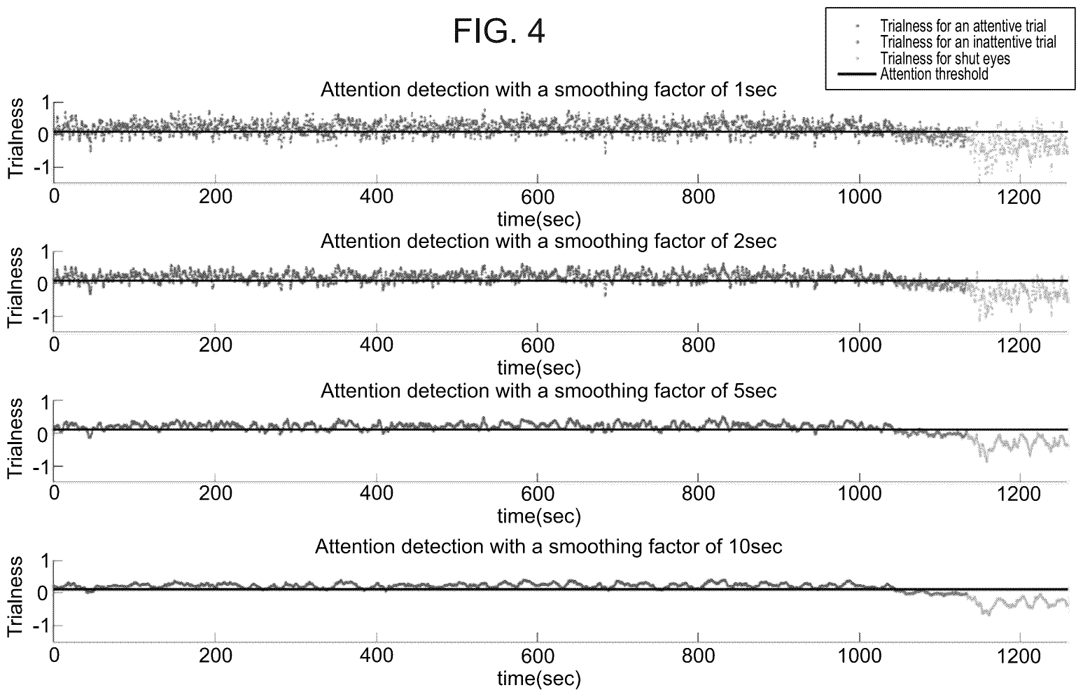

FIG. 4 shows the trialness signal obtained from a set of trialness values and smoothed with a smoothing factor (window size) of 1 second (top panel), 2 seconds (second panel), 5 seconds (third panel), and 10 seconds (bottom panel). The attention threshold is marked by a thick black line. Blue color corresponds to time intervals in which the subject was attentive to the images, red color corresponds to time intervals in which the subject was inattentive to the images, and yellow color corresponds to time intervals in which the subject was shutting the eyes. Note that by increasing the smoothing factor makes it is easier to distinguish between attentive and inattentive states. For example, at the bottom panel (smoothing factor of 10 seconds) all red points are below the attention threshold, demonstrating that for this subject, the trialness score has 100% success of detecting loss of attention within 10 seconds.

21 Subjects

21 subjects were requested to view a series of images of various categories and search for those images that contained house. The images were displayed on a computer screen in a rate of 4 Hz. 2000 trials were used for training. To test trialness accuracy, the subjects were requested again to search for houses (Attentive task, 800 trials), but also to gaze off the screen (Gaze off task, 400 trials), and engage in a distraction task (solve arithmetic problems) while looking at the screen, so they would be inattentive to the images (Inattentive task, 800 trials). The subjects had a break every 100 seconds.

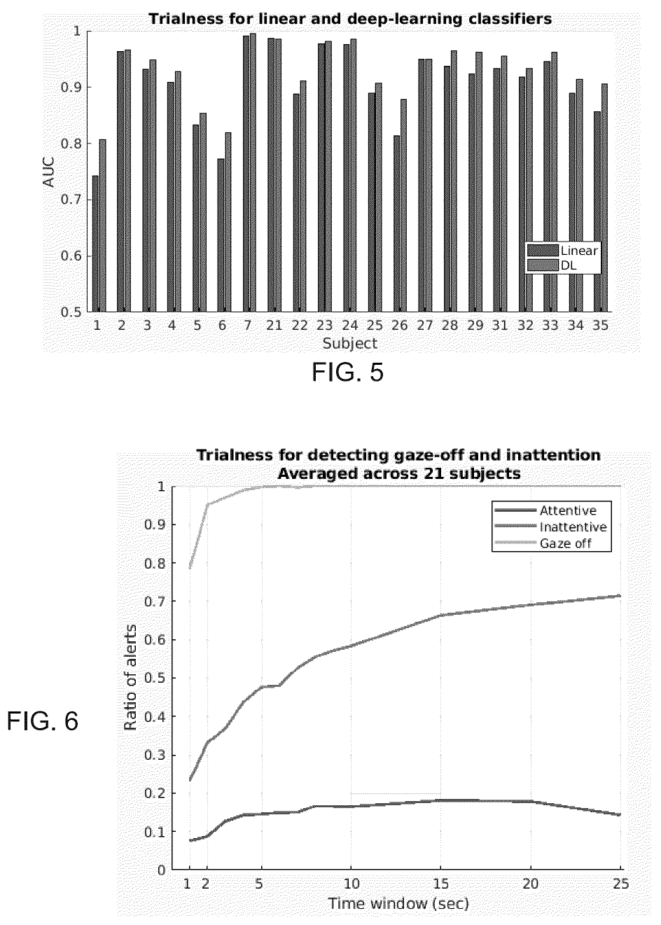

FIG. 5 shows a comparison between the accuracy of linear classifier and the deep learning (CNN, in the present example) classifier (see methods). As shown, for most of the subjects deep-learning yielded higher AUC. For the AUC calculation, the data from Attentive task was given label ‘1’ and the data from the Inattentive and Gaze off tasks was given label ‘0’.

FIG. 6 demonstrates increase in performance accuracy with data accumulation. Shown is the rate of positive decisions per condition as a function of the window size. The blue line represents false positive rate (trials falsely detected as inattentive out of all truly inattentive trials), and yellow and red lines represent true positive rate (trials correctly detected as inattentive out of all trials detected as inattentive) for Gaze-off and Inattentive, respectively. Moving along the time axis, one observes the increase in performance accuracy as more and more data is accumulated. For example, after 2 seconds it is possible to detect 95% of gaze-off cases, but only a third of inattention.

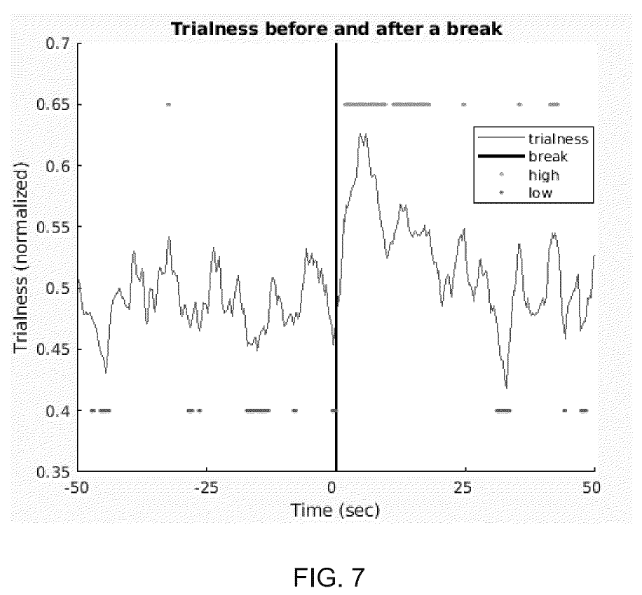

FIG. 7 shows normalized trialness scores, averaged across the 21 subjects, before (t<0) and after (t>0) a break (t=0). In order to test at which time-points the attention was shifted, a series of t-tests were conducted. In each t-test, the trialness for all subjects at a certain time was compared to the median score (0.5). Significant time points (p<0.05) are highlighted in FIG. 5 (green for high trialness, red for low trialness). As shown, after a break the subjects showed higher trialness levels. This lasted for some 20-25 seconds. Since subjects are typically more attentive after a break, FIG. 7 demonstrates that the trialness measure of the present embodiments can serve as a measure for attention.

This Example demonstrates that the trialness measure of the present embodiments is effective in detecting overt attention shifts, where subjects look away from the images or shut their eyes. This Example demonstrates that the trialness measure of the present embodiments is also effective in detecting covert attention shifts (when subjects looked at the images but where not paying attention to them), within a time period of about 15 sec on average.

### Example 2

### Estimation of Attention from Labeled EEG Data

This Example describes time-domain and frequency-domain classifiers trained based on labeled EEG data. EEG signals were collected while instructing subjects to stare at the images without performing any task (covert loss of attention). Eyes-shut data (overt) and other covert and overt inattentive tasks were also collected. The classifiers were then trained to distinguish between attentive and inattentive states. Both time-domain classifiers and frequency-domain classifiers were used.

### Methods

EEG signals were recorded from the brain, while the subjects were presented with a set of images as a visual stimulus. The EEG signals were digitized to provide EEG data, and the data were preprocessed by applying a band pass filter 1-30 Hz, and by removing artifacts. The data was segmented from −100 ms to 900 ms relative to image onset. For the frequency domain classifier, Fourier transform was applied to each segment separately, keeping 1 Hz to 30 Hz frequency bins.

The time domain classifier was trained to distinguish between attentive and inattentive time segments, and the frequency domain classifier is trained to distinguish between attentive and inattentive frequency bins.

After training, the time domain and the frequency domain classifiers were fed with EEG data obtained for the same subject, but during a different image-review session.

Time Domain Classifier

Each input segment included N EEG data samples over M channels. The classifier in this Example was a CNN having the architecture shown in FIGS. 3A-B.

Frequency Domain Classifier

The input data for a single segment included K frequency bins over M channels. In this Example, 30 frequency bins over a frequency range of 1-30 Hz were used. The classifier in this Example was a CNN having the architecture shown in FIGS. 3A-B.

### Results

7 Subjects

7 subjects were requested to perform four different tasks while a series of images of various categories was displayed on a computer screen at a rate of 4 Hz. In a first task, and search for those images that contained houses (Attentive task). In a second task, the subjects were requested gaze off the screen (Overt Inattentive task). In a third task, the subjects were requested to stare at the screen without being attentive to the displayed images (Covert Inattentive task). In a fourth task, the subjects were requested to shut their eyes (Overt Inattentive task).

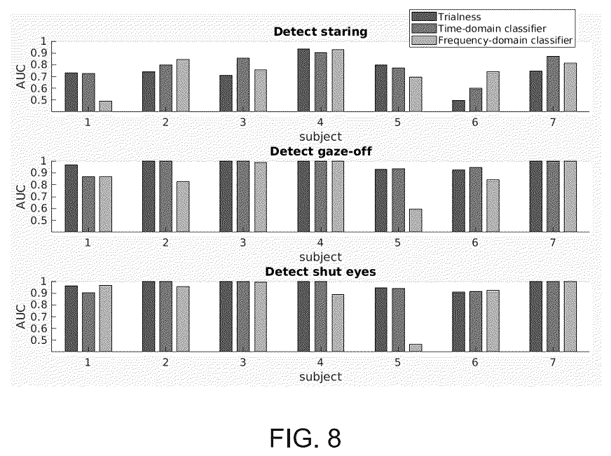

FIG. 8 shows a comparison between the trialness score (blue bars), and the scores produced by the time-domain (red bars) and frequency-domain (orange bars) CNNs trained using the labeled EEG data. Shown are AUC results, for two-second epochs (8 images), for staring inattention (top panel), gaze-off inattention (middle panel) and eyes shut inattention (bottom panel), as detected by each of the three classifiers.

FIG. 8 demonstrates that for most subjects, the trialness score is effective for detecting overt inattention (eyes shut and gaze-off) with AUC above 0.9. For covert inattention (staring), however, some subjects (subject Nos. 2, 3, 6 and 7) benefited from using the time-domain or frequency-domain classifiers.

### Example 3

### Combining Scores

### Methods

In order to combine different classifiers (Trialness, Time-domain, and Frequncy-domain, in this example), the validation data were classified using all 3 three classifiers and the AUC of each classifier was computed. For each subject, classifiers for which AUC was less than 0.1 compared to the best classifier were discarded, by assigning them a zero weight. For the remaining classifiers the following formula was used for calculating the weight:

$$\frac{A U {C}_{i} - 0.5}{{\sum }_{i = 1}^{n} ( A U {C}_{i} - 0.5 )}$$

where AUCᵢ is the AUC value of the ith classifier of a total of n classifiers.

Referring to FIG. 8, in the top panel, the AUC values of subject No. 1 for the trialness, time-domain and frequency domain classifiers are 0.733, 0.725 and 0.492, respectively. The weight of the third classifier was thus set to zero because it is smaller by more than 0.1 compared to the maximum AUC. The weights of the first two classifiers for subject No. 1 are 0.509 and 0.491. The scores of the three classifiers are then normalized to values between zero and one, then multiplied by their corresponding weights and summed. The resulting set of scores, one score per trial, was used as a predictor for the likelihood that the subject's brain was in attentive state.

The combined classifiers were tested on a cohort of 25 subjects. The subjects were requested to perform a series of tasks in 3 different days.

Day 1
(i) Shut eyes for 5 min (“Shut A”)
(ii) Look at a blank screen for 5 min (“Open A”)
(iii) Detect images of houses among 7 other categories displayed on a computer screen in a rate of 4 Hz for 10 min
(iv) Detect images with pixelated areas among regular images displayed on a computer screen in a rate of 4 Hz (“Pix A”) for 10 min
(v) Shut eyes for 5 min (“Shut B”)

Day 2
(i) Detect images with pixelated areas among regular images displayed on a computer screen in a rate of 4 Hz (“Pix B”) for 10 min
(ii) Look at a blank screen for 5 min (“Open B”)
(iii) Detect images with pixelated areas among regular images displayed on a computer screen in a rate of 4 Hz (“Pix C”) for 10 min
(iii) Stare at the screen where images are displayed in a rate of 4 Hz (“Stare”) for 5 min

Day 3
(i) Perform a 30 min Uchida-Kraepelin test, which is a paper and pencil task (adding numbers in long rows) (“UKTest”)

Attentive states were defined as tasks where the subjects were requested to detect targets (“House”, “PixA”, “PixB”, “PixC”), and all the rest of the tasks were defined as inattentive. The collected data was classified using the Trialness classifier, Time and Frequency domain classifiers and the Combined classifier to detect attentive vs inattentive states.

### Results

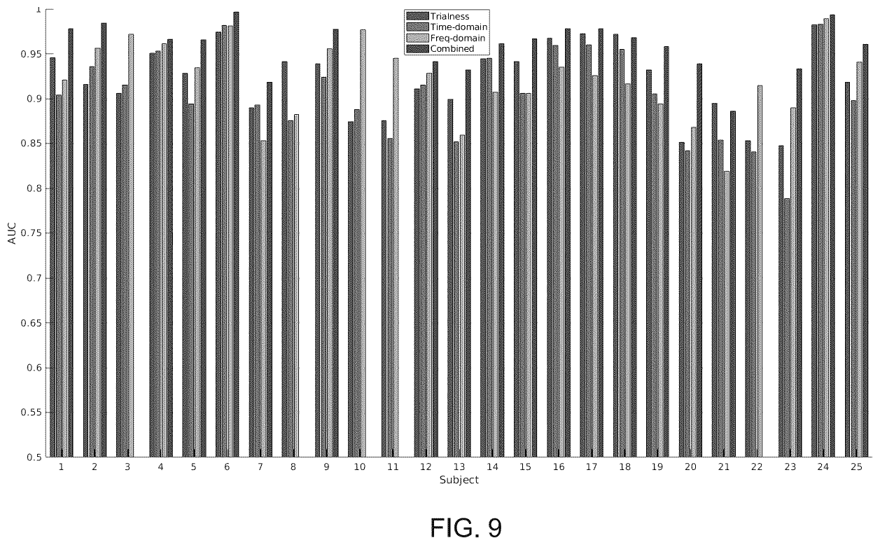

FIG. 9 shows AUC performance for detecting attentive states using the four classification methods. As shown, for 18 of the 25 subjects, the highest AUC was obtained for the combined classifier. For the other subjects, other classifiers achieved the maximum AUC.

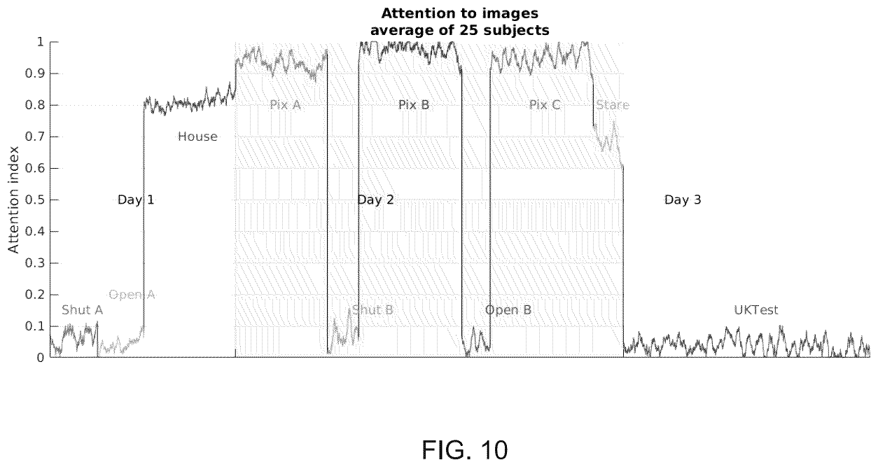

FIG. 10 shows an attention index, which is defined as the score obtained for each subject using the classifier that provided the highest AUC for this subject, averaged over the 25 subjects. FIG. 10 demonstrates the ability of the attention index to distinguish between attentive and inattentive states. This can be done by thresholding wherein when the attention index is above a predetermined threshold, the brain is in an attentive state and when the attention index is not above the predetermined threshold, the brain is in an inattentive state. In this Example, predetermined threshold can be about 0.76.

### Example 4

### Estimation of “Trialness” for Auditory Stimuli

Four medical students were requested to listen for pathologic stethoscope recordings (crackles). The data was processed in the same way as in Example 1 section 3, except that the fixed beginning windows (“true trials”) were defined from −100 ms to 185 ms (window width 285 ms) relative to the auditory stimulus onset. A trialness classifier was trained and tested for every subject separately. In addition, another classifier was trained for all the data combined.

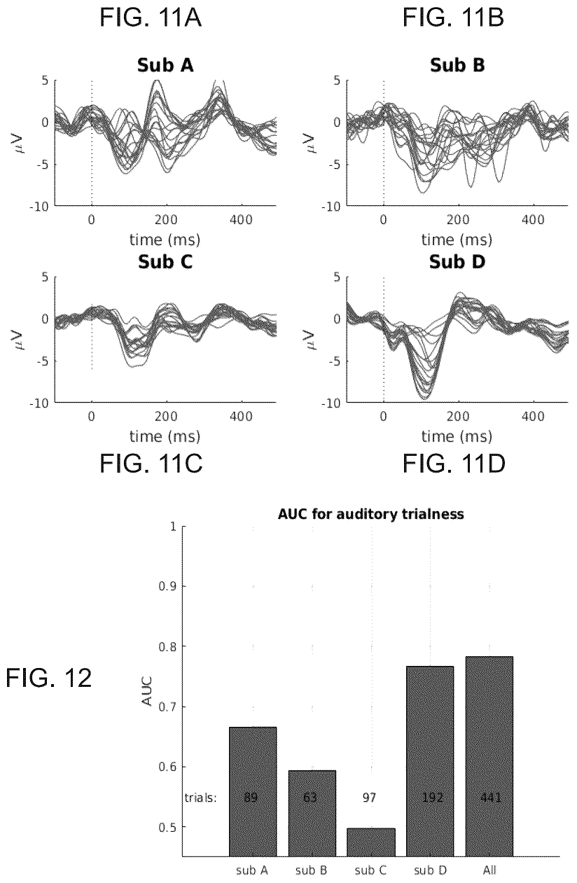

FIGS. 11A-D show the Evoked Response Potential (ERP) for each of the four subjects, and FIG. 12 shows the trialness classifier AUC. The number on the bar indicates the number of trials that were used for training the classifier.

For three subjects (Sub A, Sub B, Sub D) the performance was adequately high (0.59 to 0.76). The classifier trained on the combined data yielded a similar result (0.78). This Example demonstrate the ability of the trialness measure of the present embodiments to estimate the likelihood that the brain is in attentive state, also for the case in which the stimuli are auditory.

### Example 5

### Estimation Attention without Synchronization with Stimuli

This Example describes a technique for estimating attention in cases in which the EEG data are not synchronized with stimuli. The technique can be used for estimating the likelihood that the brain is in an attentive state while performing a task-of-interest which is not driven by stimulus. For example, the task-of-interest can be performed at random time intervals or at time intervals selected by the subject itself.

The described technique is based on a machine learning procedure of a logistic regression type. The training of the procedure is specific to the subject and also specific to the task-of-interest for which attention is to be estimated. For a given type of task-of-interest (e.g., a visual processing task, an auditory processing task, a working memory task, a long term memory task, a language processing task, multitasking, etc.), two sets of training tasks are selected. A first set includes attentive training tasks that are of the same type as the task-of-interest, and a second set include inattentive training tasks that are of a different type than the task-of-interest. The training tasks in the first set mimic the task-of-interest, and the training tasks in the second set mimic loss of attention for performing the task-of-interest.

This Example describes the procedure for two types of task-of-interest: a task that relates to data entry, and a task that relates to image annotation. For performing the task that relates to data entry, the subject is requested to locate specific data items and type them into a form. For performing the task that relates to image annotation the subject is requested to mark bounding boxes around specific types of objects in images.

### Methods

### Tasks

In this Example, the following tasks were used for generating the training data for the logistic regression.

Data Entry

The subject was presented with an image containing different numerical data items (prices, review scores, numbers of reviewers for different products). In a different session, the subject was presented with the table containing other types of data items (dates, names, salaries). The subject was asked to enter specific data values into specific data field within a form.

Game

The subject was presented with an animation of falling numbers on a screen, and was requested to type the numbers before they reached the bottom of the screen.

Mind Wandering

Same as the Game task above, but while watching falling numbers, subjects had to imagine their next vacation, or last weekend.

Reading

The subject was presented with a paragraph on a randomly selected topic for reading, and was requested to rate the level of interest on the topic.

Sustained Attention Response Task (SART)

The subject was presented with a sequence of digits on a screen, and was requested to press a corresponding digit key on a keyboard after each displayed digit, except when the digit was 3. The task was deliberately boring, and was selected so that it was difficult to maintain concentration. Errors were measured.

Image Annotation

The subject was presented with a series of images on a screen, and was requested to draw on the screen bounding boxes around specific objects (e.g., large vehicles, bottles) within the images.

Eyes Open

The subject was requested to rest with eyes open.

Eyes Shut

The subject was requested to rest with eyes closed.

### Protocol

19 subjects participated in the experiment. The subjects came for two visits. In a first visit, the subjects were requested to perform the Data entry, Game, Mind Wandering, Reading, SART, Eyes open, Eyes shut, and Image annotation tasks. in a second visit, the subjects were requested to perform the Reading, Data entry, Eyes shut, Eyes Open, and Image annotation tasks.

### Data Collection and Labeling

The EEG data were collected and segmented into segments of 2 seconds using a sliding window of ⅓ seconds and ⅚ seconds overlap between windows. The input data for the classification included 2D data segments of N time points over M channels, per data segment.

Data collected in the first visit were defined as training datasets, and data collected in the second visit were defined as validation datasets.

The segments were labeled with “0” or “1” depending on the task performed within the respective segment, and depending on the task-of-interest. Specifically, when the task-of-interest was Data Entry, segments during which the subject performed the Data Entry task were labeled “1” and segments during which the subject performed any other task were labeled “0”, when the task-of-interest was Image Annotation, segments during which the subject performed the Image Annotation task were labeled “1” and segments during which the subject performed any other task were labeled “0”.

### Data Analysis

In this Example the machine learning procedure was trained to provide a score that estimates the likelihood that the brain of a specific subject is attentive to the specific task-of-interest, defining all other activities that the subject may be engaged with as background tasks. This score is referred to herein as “task-specific attention.” In this Example the task-specific attention has a value in the range [0, 1].

The machine learning procedure was trained separately for each subject and separately for each task-of-interest.

The segmented EEG data were filtered by a bandpass filter of 1-45 Hz. A vector of classification features was extracted for each data segment. Depending on the number of electrodes, different amounts of features were calculated, as some features are channel-specific and others look for inter-channel features. For example, for a 7-electrode EEG system, there were 723 classification features and one label.

The classification features used in this Example are summarized in Table 5.1, below, where M is the number of channels (M=7, in this Example).

TABLE 5.1

| Feature type | Number of features |
|---|---|
| Mean/min/max values of each channel in the time | 3M |
| window | |
| Change in mean/min/max of signal (per channel) | 3M |
| between first and second half-windows | |
| Mean/min/max of signal (per channel) in all quarter- | 3M*4 |
| windows | |
| Change in mean/min/max of signal (per channel) | 3M * (4 − 1)! |
| between all quarter-windows | |
| Standard deviation per channel for time window | M |
| Change in standard deviation per channel for every | M |
| half-window | |
| Skewness and kurtosis per channel for time window | 2M |
| Covariance matrix across channels | M + (M − |
| | 1) + . . . + 1 |
| Eigenvalues of covariance matrix | M |
| FFT values (From 1.5 Hz to 25 Hz, jumps of .5) per | 48M |
| channel for time window | |
| Top 10 frequencies per channel | 10M |
| Blinks per minute and vertical eye movements per | 2 |
| minute as detected from EEG | |

These feature vectors were converted to Z-scores in accordance with the distribution of feature scores in the training data. The conversion procedure was saved for use also on test data.

A logistic regression procedure was trained on the Z-sores of the training set using the labels assigned to each segments, providing a trained logistic regression function defined by a set of learned coefficients that respectively correspond to the set of features that form each of the feature vectors. The Task-Specific Attention for a given segment of the validation dataset of a particular subject was calculated by applying the trained logistic regression function, including the coefficients as learned for the particular subject, to the feature vector of the given segment.

### Results

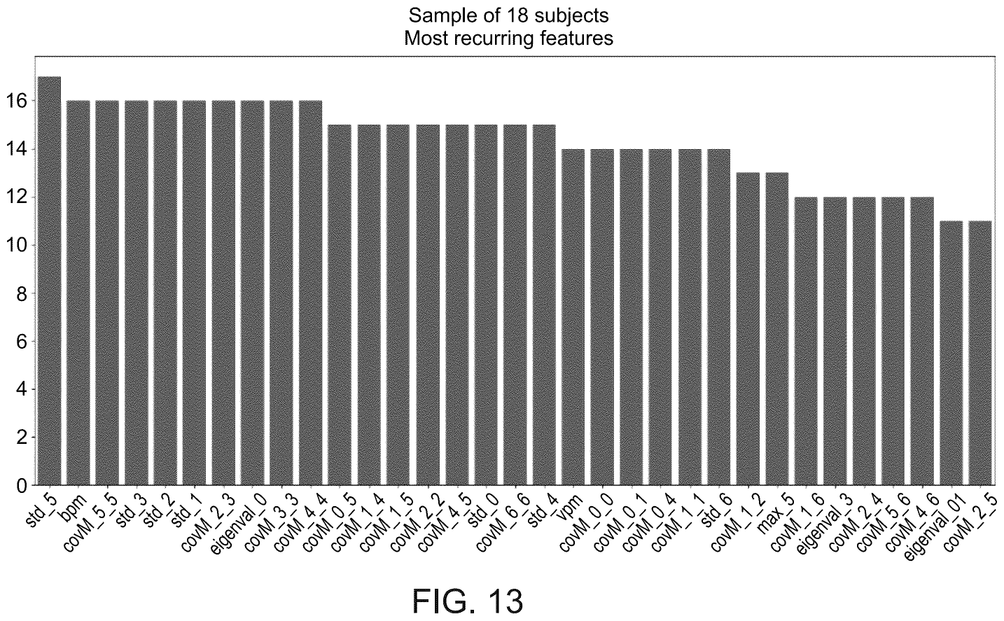

FIG. 13 shows 33 features that were found to be influential on the logistic regression function for a pool of 18 subjects. The following abbreviations are used in FIG. 13:
std: Standard deviation of signal
bpm: Blinks per minute
vpm: Vertical eye movements per minute
covM: Covariance (of 2 channels)
eigenval: Eigenvalue of covariance matrix
max: Maximum value of signal
{Feature}_X: The X indicates the index of the relevant electrode (channel)
{Feature}_X_Y: For features that depend on interaction between 2 electrodes of index X and Y.

The trained logistic regression function as obtained for each subject was applied to the segments of the validation dataset, and was then evaluated for correct detection of the states based on the assigned labels.

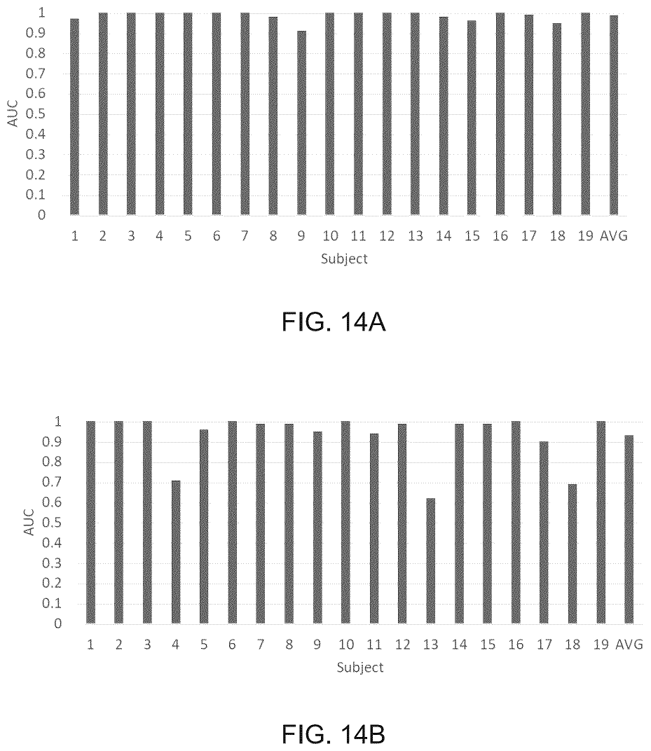

FIGS. 14A and 14B show AUC values of the task-specific attention, when the task-of-interest was defined as Data Entry (FIG. 14A) and Image Annotation (FIG. 14B), for 19 subjects. Also provided is an average AUC value obtained by averaging over all subjects. As shown, on the average, all classifiers reach AUC of more than 0.9.

### Example 6

### Estimation of Concentration

The Inventors found that EEG patterns that are typical to general concentration can be distinguished from EEG patterns that are typical to a specific task. This Example describes a classifier trained to detect whether or not the subject is concentrated, irrespectively of the specific task the subject is performing.

### Methods

The tasks and the protocol were the same as in Example 5.

### Data Collection and Labeling

The EEG data were collected and segmented into 2s segments (stride=0.5 s, and 75% overlap).

The labels used in this Example are summarized in Table 6.1, below.

TABLE 6.1

| Task | Label |
|---|---|
| Image Annotation | 1 |
| Data Entry | 1 |
| Game | 1 |
| Reading | 1 |
| Mind Wandering | 0 |
| Eyes open | 0 |
| Eyes shut | 0 |
| SART | 0 |

Thus, a segment was labeled non-specifically with a “1” for all tasks at which the subject was requited to provide an input that is positively correlated to the goal of the task (and is therefore indicative of the subject's level of concentration). All other tasks were considered as background. Note that SART is considered a background task since the count was of the number of errors.

Data collected in the first visit were defined as training datasets, and data collected in the second visit were defined as validation datasets (see Example 5: Protocol).

### Data Analysis

For classification, a CNN was used. In this Example, the architecture of the CNN was the same as shown in FIGS. 3A and 3B. A median filter was then applied to the classification scores generated by the CNN.

During training, each segment was labeled according to the task performed during the segment, to compose a vector of length N (number of segments), denoted Y_train.

Each segment was subjected to preprocessing which included detrending, applying Fourier transform (n=300), converting the spectrum to absolute value, and clipping at 45 Hz. This provided a dataset matrix, X_train, of dimension N by M by K, where M is the number of channels and K is the number of frequency bins.

The CNN was trained using gradient descent (Adam Optimizer, learning rate of 10⁻⁴).

### Results

The segments of the validation dataset were fed into the trained CNN as obtained for each subject, and the scores provided by the CNN were evaluated for correct detection of the states based on the assigned labels.

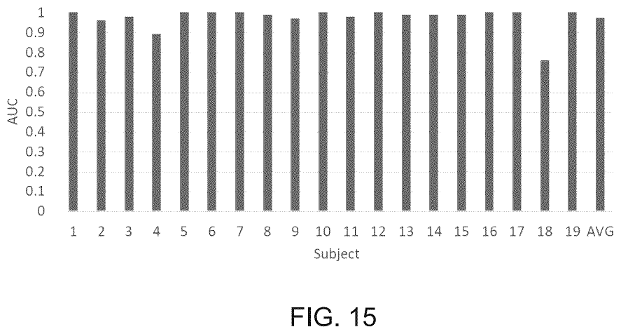

FIG. 15 show AUC values of the obtained scores for 19 subjects. Also provided is an average AUC value obtained by averaging over all subjects. As shown, on the average, all classifiers reach AUC of more than 0.9, demonstrating that the procedure of the present embodiments is capable of estimating the likelihood that a subject is concentrated, irrespectively of the specific task the subject is performing.

### Example 7

### Estimation of Awareness State

The Inventors found that EEG patterns that are typical to a brain awareness state can be distinguished from other EEG patterns by clustering. This Example describes a clustering procedure which can detect whether or not the subject's brain is in an awareness state.

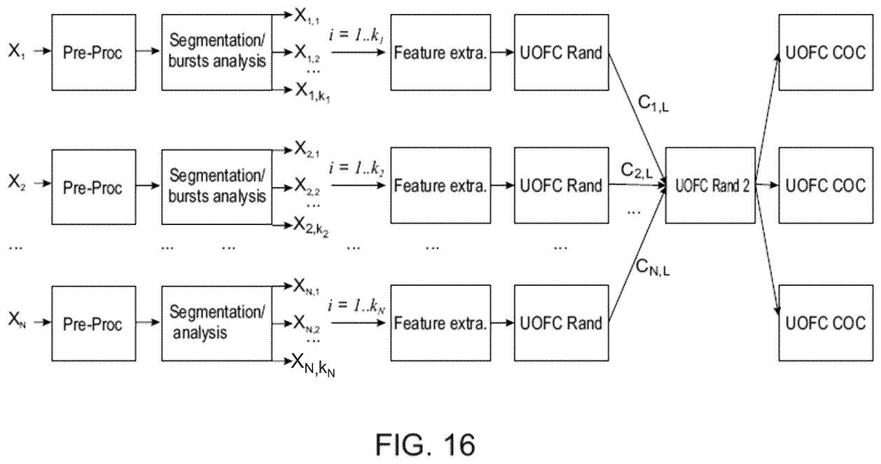

Given N ongoing EEG matrices Xₙ n=1, 2 . . . N∈R^{m}^{ₙ}^{xe} where mₙ is the number of samples for the n^{th} subject and e is the number of electrodes, a clustering procedure was executed. The procedure will now be described with reference to FIG. 16.

The data matrix of each subject is preprocessed by applying bandpass filter and removing blinks and artifacts. Segmentation was then applied to the data matrix of each subject. In this Example, two types of segmentations were employed.

In a first type of segmentation, the matrix was segmented into 2 second windows, with 1 second overlap, resulting in kₙ segments for the n^{th} subject.

In a second type of segmentation, referred to herein as burst analysis, a Hilbert transform was applied to each channel of the matrix to obtain an energy band envelope of the channel. Energy above a predetermined threshold was considered as a “burst”, and segments were defined according the detected bursts.

Features were then extracted from each of the segments and each channel. When the first type of segmentation was employed, the features were the energy in the Alpha, Beta, Delta, Theta and Gamma bands. These features were extracted using Fast Fourier Transform (FFT). When the second type of segmentation was employed, the features were, for each of the Alpha, Beta, Delta, Theta and Gamma frequency bands, the peak amplitude of the burst in the respective frequency band, the area under the envelope curve in the respective frequency band, and the duration of the burst in the respective frequency band. The number of features that are extracted for each segment is denoted D, and so each segment is assigned with a D-dimensional feature vector.

A first Unsupervised Optimal Fuzzy Clustering (UOFC) procedure was then applied to the features of each subject, to provide L clusters for each subject, and a total of NL clusters (N being the number of subjects in this Example). The cluster centers were initialized randomly. The D-dimensional central feature vector of the ith cluster that was obtained by the UOFC for the nth subject is denoted C_{n,i}.

An additional UOFC procedure was applied to the D-dimensional centers C_{n,i} (n=1, . . . , N, i=1, . . . , L), providing a set of L centers of the D-dimensional centers, denoted {COC}. A further UOFC procedure was then applied to the features of each subject, to provide, again, L clusters for each subject, and a total of N·L clusters, except that in the further UOFC the respective element of the set {COC} was used as an initializer for each of the cluster centers, instead of the random initializer used in the first UOFC procedure. In addition, the L cluster centers can also be added as features to the set of original features for the further UOFC re-clustering procedure.

The output of the further UOFC was a membership matrix for each subject that represented the membership (0-1) of a segment to a given cluster. The membership value was defined to be proportional to 1/d_{i,j}, where d_{i,j}, is the distance of the jth segment features to the ith cluster. In this Example, an exponential metric (e\hat{ }(−d_{i,j} 2) was used for measuring the distance.

For each subject, the average membership of the ith cluster to the task associated with high fatigue, or mind wandering was calculated, and the cluster that yields the highest average membership value was defined as a “fatigue cluster”. Note that the selected cluster was also affected by the eyes shut traits of the other subjects due to the COC.

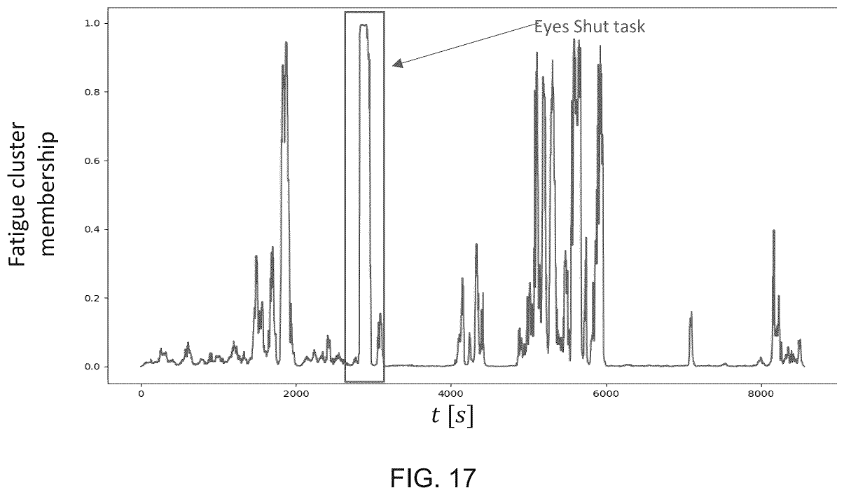

FIG. 17 shows the cluster memberships of the segments for the cluster associated with the energy in the alpha band. The membership of the Eyes Shut segment, which is indicative of a fatigue state of the brain, is the highest, demonstrating that the clustering procedure of the present embodiments is capable of detecting segments during which the brain is in a fatigue state.

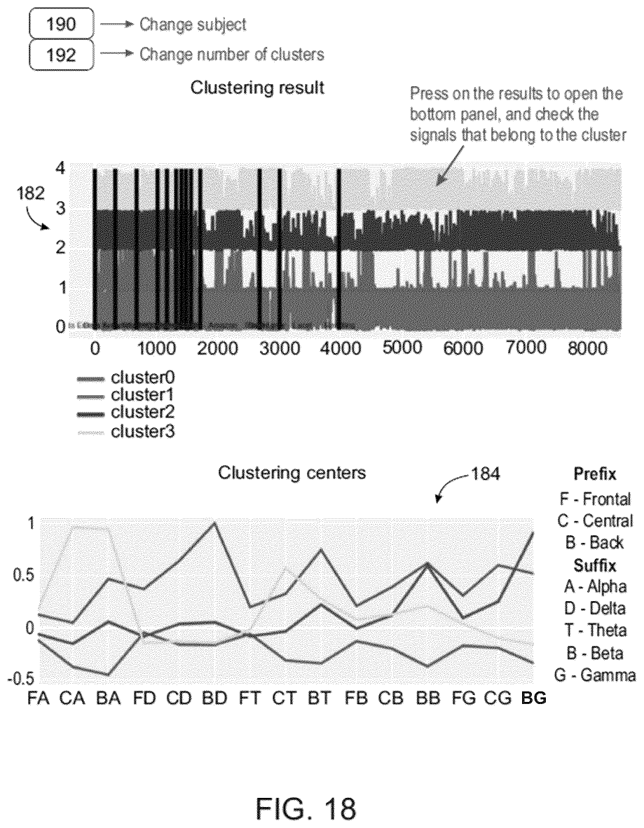

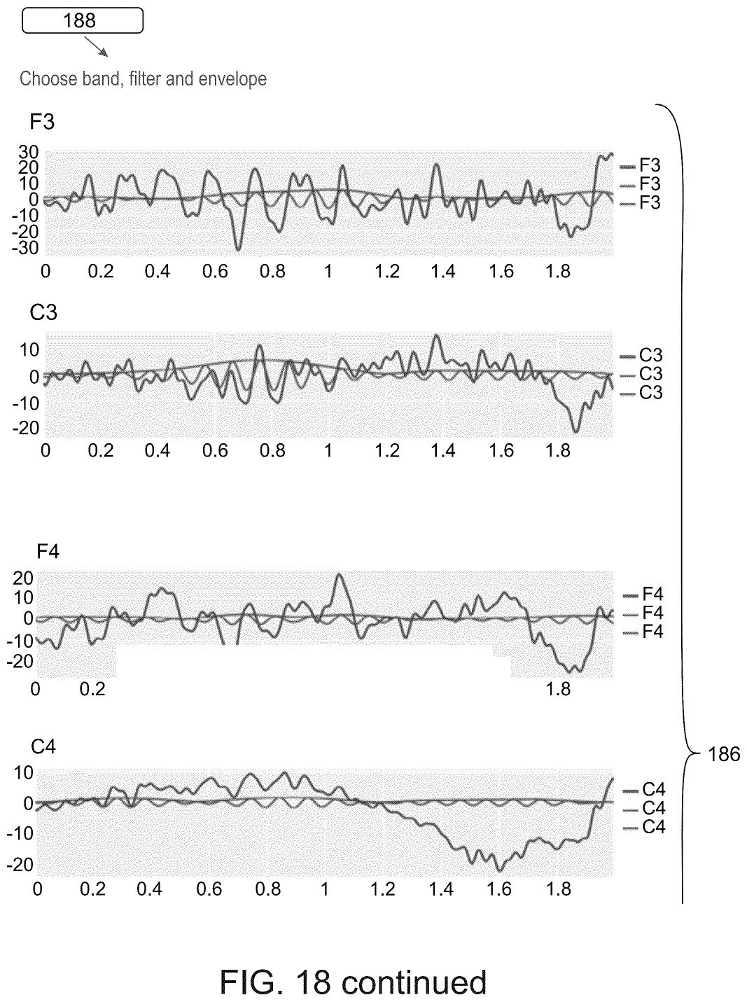

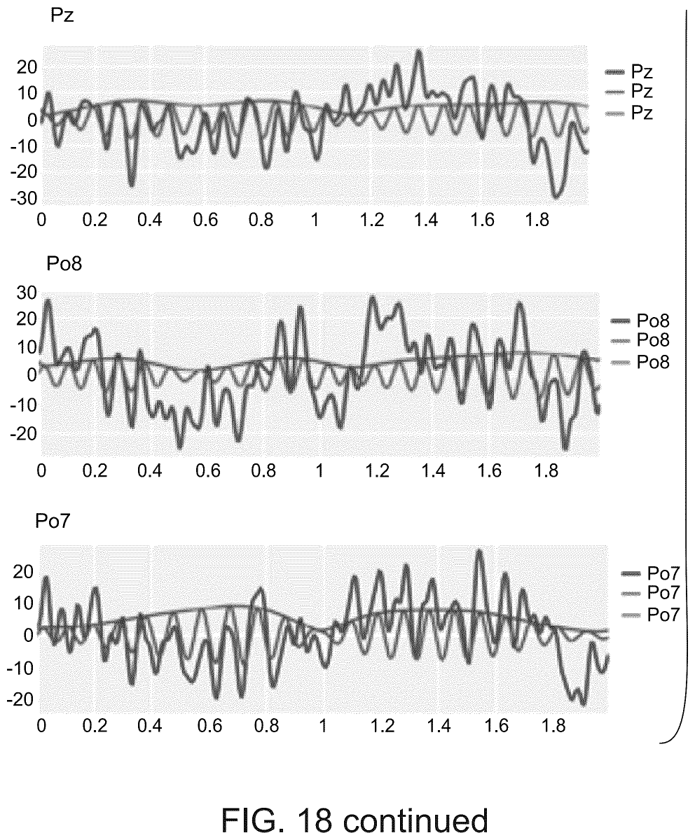

A representative example of a GUI presenting the output of the clustering procedure is illustrated in FIG. 18. The upper left region 181 shows clusters membership as a function of time. In this example, 4 clusters were used, each cluster is shown in different color (yellow, blue, green, red). The upper right region 184 shows clustering centers for each of the clusters. The bottom region 186 shows raw data and detected features (in this example envelopes of alpha band) for all channels (in this example 7 channels). Several controls can be provided on the GUI. One control 188 allows the operator to select a band, a filter and an envelope, another control 190 allows the operator to select the subject, and another control 192 allows the operator to selected the number of clusters.

The clustering procedure described in this Example was evaluated on the dataset of the 19 subjects presented Examples 5 and 6 above. The tasks were labeled such that eyes shut represented a fatigue state, to simulate a situation in which the person is sleepy. Segments during which the eyes were closed were therefore labeled “1”. Segments with eyes open during a break after a long working task, when a person was not concentrated were also labeled “1”. Segments during which other tasks were performed were labeled “0”.

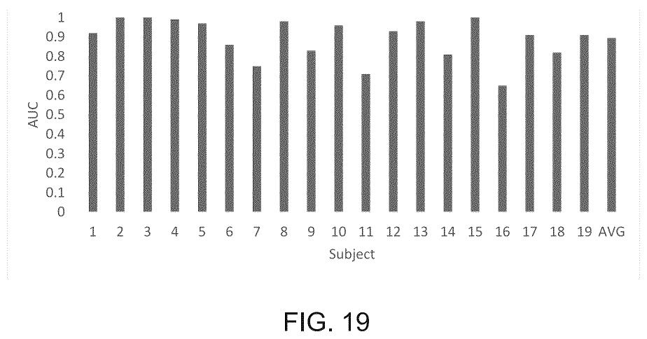

FIG. 19 shows AUC values obtained for 19 subjects. Also provided is an average AUC value obtained by averaging over all subjects. As shown, on the average, the AUC values are more than 0.9, demonstrating that the clustering procedure of the present embodiments is capable of estimating the awareness state of the brain of a subject.

### Example 8

### Mind Wandering

The Inventors found that EEG patterns that are typical to a mind wandering state can be distinguished from other EEG patterns. This Example describes a machine learning procedure which can detect whether or not the subject's brain is in a mind wandering state.

EEG signals were collected from 10 subjects while the subjects performed a SART task (see Example 5, Methods).

The EEG signal was preprocessed as further detailed hereinabove, and was then filtered to canonical EEG bands (alpha, beta, gamma, and theta). The envelope signal of each canonical frequency band was extracted.

From every no-go onset (triggered by the appearance of the digit “3” on the screen), a segment of EEG signal was collected. The segment was 4 seconds in duration such that the end of the segment was 200 ms before the onset. The 200 ms offset ensured that there was no leakage from the EEG the signal after the onset into the segment. Segments from the filtered and envelope signals were collected similarly, and were used as extra channels.

The 4s segments were considered as trials, and were labeled “1” if the subject failed in the no-go task, namely responded to the onset (denoted as “commission error”), and “0” if the subject succeeded in the no-go task, namely did not respond to the onset (denoted as “correct rejection”). Trials were collected from multiple subjects and were mixed together to form X_train matrix, and a Y_train vector containing the labels.

The X_train matrix and Y_train vector were used to train a neural network using gradient descent (Adam optimizer, learning rate of 10⁻⁵). The model was fine tuned with personal data of the subject. To this end the neural network was trained with a small dataset composed only from trails from the particular subject, using a lower learning rate and while freezing the 2 bottom layers of the network.

An ensemble of five neural networks was formed, where the neural networks differ from each other by excluding different set of subjects from the train set. The subjects excluded from the train set were used as validation set for evaluation and early stopping. Neural networks which achieve AUC score of above 0.65 on a validation set made only from trials of the particular subject formed the final ensemble.

For prediction, the EEG signal was segmented to 4s segments (sliding window, stride of 0.5s, i.e. 75% overlap). Each segment was feed-forwarded in each of the neural networks that compose the ensemble, producing an ensemble of scores, one for each neural network. The average of the ensemble of scores was defined as the score of the segment. The scores were aligned such that the score at time t corresponds to the 4s window that ends at time t. This procedure produced a mind wandering score signal sampled at 2 Hz, whose first 7 values are zeros. The first non-zero value (at the 8th index) correspond to the time window t=[0 . . . 4]s. The mind wandering score signal was then smoothed with a Gaussian filter (std=3, n_samples=10), and the time periods during which the mind wandering score signal was above a predetermined threshold (0.7 in this Example) were defined as mind wandering time-periods.

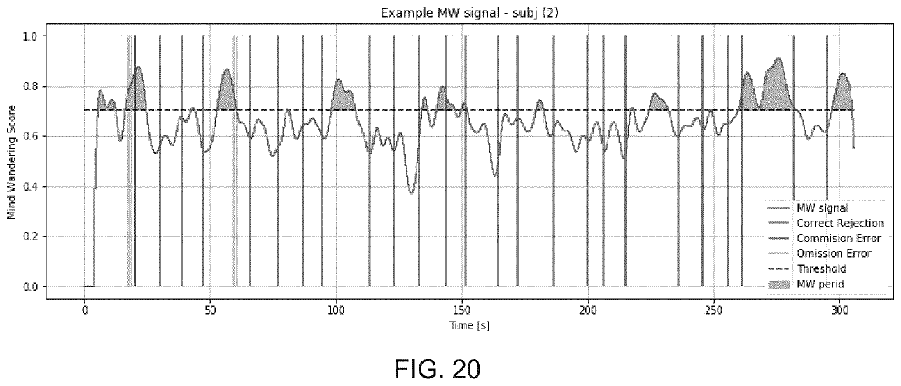

A representative example of a mind wandering signal for subject No. 2 is shown in FIG. 20.

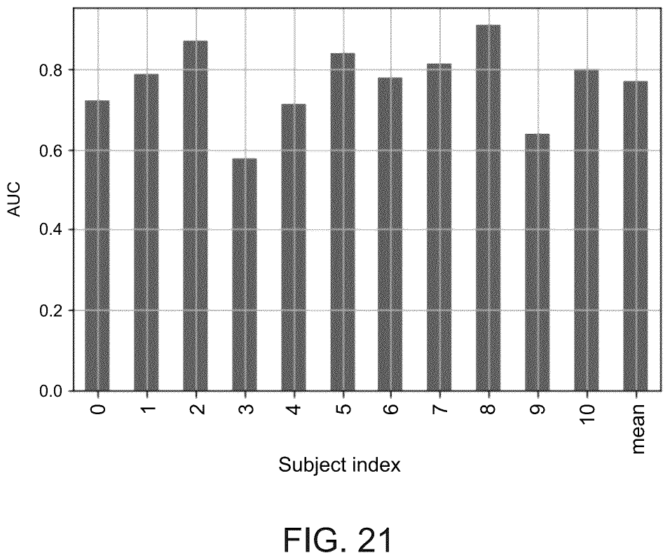

FIG. 21 shows the AUC of the commission error prediction as calculated for each of the 10 subjects. As shown, on the average, the AUC values are close to 0.8, demonstrating that the procedure of the present embodiments is capable of estimating the likelihood that a brain of a subject is in a mind wandering state.

### Example 9

### Exemplary Combined Output

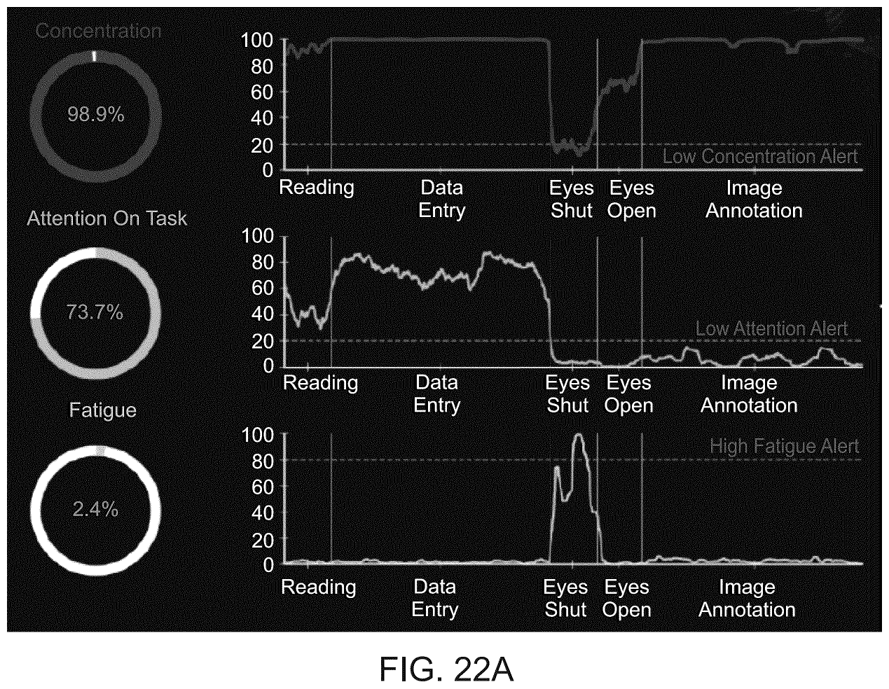

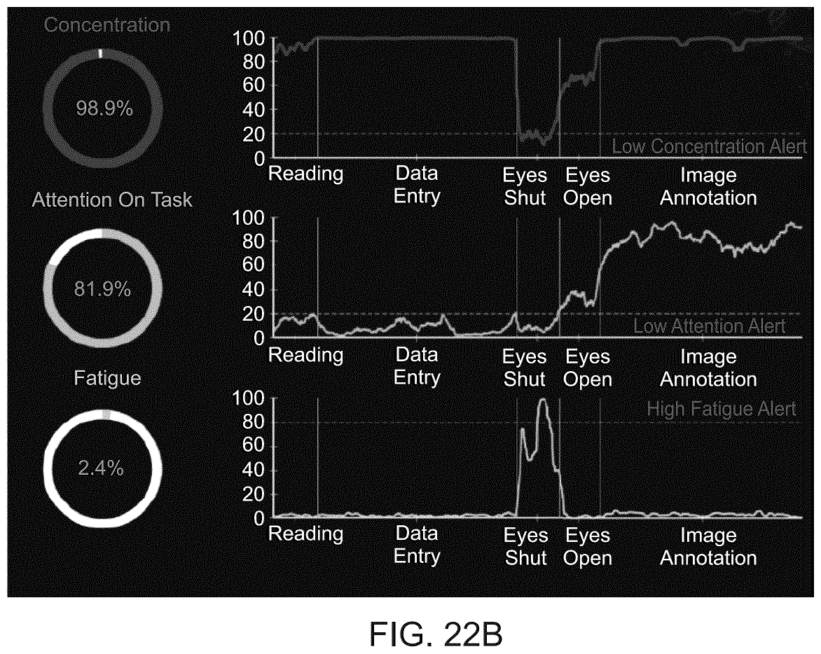

Exemplary combined outputs for estimation of brain states are shown in FIGS. 22A and 22B for the Data Entry task (FIG. 22A) and the image annotation task (FIG. 22B). The time axis also shows other tasks including the Reading task, the Data Entry task, the Eyes Shut task, the Eyes Open task, and the Image Annotation task, see Example 5, method, for a description of these tasks. The brain states that are estimated in each of FIGS. 22A and 22B are concentration (top), task-specific attention (middle), and fatigue (bottom), see Examples 5, 6 and 7 for a description of the procedures employed for the estimation of these states. As shown the concentration score is high during Reading, Data Entry and Image Annotation and is Low during inattentive tasks of Eyes Open and Eyes Shut. The task-specific attention is high in segments during which the user was engaged in the task-of-interest, and low otherwise.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of estimating attention, comprising:
receiving encephalogram (EG) data corresponding to signals collected from a brain of a subject synchronously with stimuli applied to the subject, the EG data being segmented into a plurality of segments, each corresponding to a single stimulus;
dividing each segment into a first time-window having a fixed beginning, and a second time-window having a varying beginning, said fixed and said varying beginnings being relative to a respective stimulus; and
processing said time-windows to determine the likelihood for a given segment to describe an attentive state of the brain.

2. The method according to claim 1, wherein said varying beginning is a random beginning.

3. The method according to claim 1, further comprising receiving additional EG data collected from a brain of a subject while deliberately being inattentive for a portion of said stimuli, said additional EG data also being segmented into a plurality of segments, each corresponding to a single stimulus;
processing said segments of said additional EG data to determine an additional likelihood for a given segment to describe an attentive state of the brain; and
combining said likelihood and said additional likelihood.

4. The method according to claim 3, comprising representing each segment of said additional EG data as a time-domain data matrix, wherein said processing comprises processing said time-domain data matrix.

5. The method according to claim 3, comprising representing each segment of said additional EG data as a frequency-domain data matrix, wherein said processing comprises processing said frequency-domain data matrix.

6. The method according to claim 3, comprising representing each segment of said additional EG data as a time-domain data matrix and as a frequency-domain data matrix, wherein said processing comprises separately processing said data matrices to provide two separate scores describing said additional likelihood, and wherein said combining comprises combining a score describing said likelihood with said two separate scores describing said additional likelihood.

7. The method according to claim 1, further comprising receiving additional physiological data, and processing said additional physiological data, wherein said likelihood is based also on said processed additional physiological data.

8. The method according to claim 7, wherein said additional physiological data pertain to at least one physiological parameter selected from the group consisting of amount and time-distribution of eye blinks, duration of eye blinks, pupil size, muscle activity, movement, and heart rate.

9. The method according to claim 1, comprising extracting spatio-temporal-frequency features from the segments, and clustering said features into clusters of different awareness states.

10. The method according to claim 9, wherein said awareness states comprise at least one awareness state selected from the group consisting of a fatigue state, an attention state, an inattention state, a mind wandering state, a mind blanking state, a wakefulness state, and a sleepiness state.

11. The method according to claim 1, wherein said first time-window has a fixed width.

12. The method according to claim 1, wherein said second time-window has a fixed width.

13. The method according to claim 1, wherein each of said first and said second time-windows has an identical fixed width.

14. The method according to claim 1, wherein said second time-window has a varying width.

15. The method according to claim 1, wherein said processing comprises applying a linear classifier.

16. The method according to claim 1, wherein said processing comprises applying a non-linear classifier.

17. The method according to claim 16, wherein said non-linear classifier comprises a machine learning procedure.

18. A method of determining a task-specific attention, comprising:
receiving encephalogram (EG) data corresponding to signals collected from a brain of a subject engaged in a brain activity over a time period, the time period comprising intervals at which said subject performs a task-of-interest and intervals at which said subject performs background tasks;
segmenting said EG data into partially overlapping segments, according to a predetermined segmentation protocol independent of said activity of said subject;
assigning each segment with a vector of values, wherein one of said values identifies a type of task corresponding to an interval overlapped with said segment, and other values of said vector are features which are extracted from said segment;
feeding a first machine learning procedure with vectors assigned to said segments, to train said first procedure to determine a likelihood for a segment to correspond to an interval at which said subject is performing said task-of-interest; and
storing said first trained procedure in a computer-readable medium.

19. The method according to claim 18, wherein at least one value of said vector is a frequency-domain feature.

20. The method according to claim 18, wherein said first machine learning procedure is a logistic regression procedure.

21. The method according to claim 18, wherein said EG data is arranged over M channels, each corresponding to a signal generated by one EG sensor, and wherein said vector comprises at least 10M features.

22. The method according to claim 18, wherein said task-of-interest is selected from a first group consisting of tasks comprising a visual processing task, an auditory processing task, a working memory task, a long term memory task, a language processing task, and any combination thereof.

23. The method according to claim 22, wherein said task-of-interest is one member of said first group, and said background tasks comprise all other members of said first group.

24. The method according to claim 18, comprising calculating a Fourier transform for each segment, and feeding a second machine learning procedure with Fourier transform to train said second procedure to determine a likelihood for a segment to correspond to an interval at which said subject is concentrated.

25. A method of determining awareness state, comprising:
receiving encephalogram (EG) data corresponding to signals collected from a brain of a subject engaged in a brain activity over a time period;
segmenting said EG data into segments according to a predetermined protocol independent of said activity of said subject;
extracting classification features from said segments, and clustering said features into clusters;
ranking said clusters according to an awareness state of said subject.

26. A method of determining awareness state of a particular subject within a group of subjects, the method comprising:
for each subject of said group receiving encephalogram (EG) data, extracting classification features from said data, and clustering said features into a set of L clusters, each being characterized by a central vector of features, thereby providing a plurality of L-sets of central vectors, one L-set for each subject;
clustering said central vectors into a L clusters of central vectors;
for said particular subject, re-clustering said classification features, using centers of said L clusters of central vectors as initializing cluster seeds, and ranking said clusters according to an awareness state of said subject.

27. The method of claim 26, comprising supplementing said classification features by said centers of said L clusters of central vectors, prior to said re-clustering.

28. The method according to claim 26, comprising segmenting said EG data into segments according to a predetermined protocol independent of said activity of said subject.

29. The method according to claim 25, wherein said predetermined protocol comprises a sliding window.

30. The method according to claim 25, wherein said predetermined protocol comprising segmentation based only on said EG data.

31. The method according to claim 30, wherein said segmentation is according to energy bursts within said EG data.

32. The method according to claim 31, wherein said segmentation is adaptive.

33. The method according to claim 25, wherein said ranking is based on membership level of segments of said EG data to said clusters.

34. The method according to claim 25, wherein said awareness states comprise at least one awareness state selected from the group consisting of a fatigue state, an attention state, an inattention state, a mind wandering state, a mind blanking state, a wakefulness state, and a sleepiness state.

35. A method of determining mind-wandering or inattentive brain state, comprising:
receiving encephalogram (EG) data corresponding to signals collected from a brain of a subject engaged in a brain activity over a time period, the time period comprising intervals at which said subject performs a no-go task;
segmenting said EG data into segments, each being encompassed by a time interval which is devoid of any onset of said no-go task;
assigning each of said segments with a label according to a success or a failure of said no-go task in response to an onset immediately following said segment;
training a machine learning procedure using said segments and said labels to estimate a likelihood for a segment to correspond to a time-window at which said brain is in a mind wandering or inattentive state; and
storing said trained procedure in a computer-readable medium.

36. A method of estimating attention, comprising:
receiving encephalogram (EG) data corresponding to signals collected from a brain of a subject synchronously with stimuli applied to the subject, the EG data being segmented into a plurality of segments, each corresponding to a single stimulus;
accessing a computer readable medium storing a set of machine learning procedures, each being trained for estimating attention specifically for said subject, and being associated with a parameter indicative of a performance of said procedure;
for each machine learning procedure of said set, feeding said procedure with said plurality of segments, and receiving from said procedure, for each segment, a score indicative of a likelihood for said segment to describe an attentive state of said brain, thereby providing, for each segment, a set of score;
combining said scores based on said parameters indicative of said performances, to provide a combined score; and
generating an output pertaining to said combined score.

37. A computer software product, comprising a computer-readable medium in which program instructions are stored, which instructions, when read by a data processor, cause the data processor to execute the method according to claim 1.
